(19) United States
(12) Patent Application Publication
(10) Pub. No.: US 2007/0178150 A1
(43) Pub. Date: Aug. 2, 2007

(54) Pharmaceutical compositions

(76) Inventors: Prasad Adusumilli, Parsippany, NJ (US); Chungbin Kim, Parsippany, NJ (US); Stanley J. Itech, Parsippany, NJ (US); Naresh I. Mehta, Parsippany, NJ (US); Dara L. Dinner, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/583,521

(22) PCT Filed: Jul. 9, 2004
(86) PCT No.: PCT/US04/22082

§ 371 Date: Jan. 9, 2006

Related U.S. Application Data
(60) Provisional application No. 60/486,033, filed on Jul. 10, 2003.

(51) Int. Cl. A61K 9/20

(52) U.S. Cl. 424/464; 424/466

(57) ABSTRACT

A pharmaceutical composition in the form of a chewable tablet for the suppression of gastric reflux comprising an alginic acid or salt thereof, a water-soluble carbonate radical precursor, a calcium salt, a first bulk sweetener, and a binding agent. The calcium salt and either or both of the bulk sweetener and the binding agent may be blended via any of wet granulation, spray drying, or compression processes prior to admixture with the alginic acid and the carbonate radical precursor.

### FIELD OF THE INVENTION

The present invention relates to pharmaceutical compositions, and their use in the treatment of gastric reflux.

### BACKGROUND OF THE INVENTION

Various treatment for the treatment and/or suppression of gastric acid reflux have included the use of antacids, both liquid and solid as well as the proton pump inhibitors and H₂ antagonists, alone or in combination thereof. Such dosage preparations include compositions containing alginic acid, antacid materials and bicarbonates such as may be found in U.S. Pat. No. 5,888,540; U.S. Pat. No. 5,112,813; U.S. Pat. No. 5,254,591; U.S. Pat. No. 5,036,057; U.S. Pat. No. 4,869,902; U.S. Pat. No. 4,414,198; U.S. Pat. No. 4,613,497; U.S. Pat. No. 4,140,760; WO 01/10405; GB 2 298 365; and GB 2 349 570, whose disclosures are incorporated herein by reference in their entirety.

Prior preparations containing alginic acid or a salt thereof, such as sodium alginate, and a bicarbonate salt, such as sodium bicarbonate, have been known upon chewing in the mouth, to cause the alginic acid to react with the bicarbonate salt, and in the presence of saliva in the buccal cavity, to produce carbon dioxide and a highly viscous solution of, in this instance, sodium alginate. The result of this reaction is a mixture not generally considered acceptable or palatable to the consumer being in the form of a foaming, viscous, sticky mass which has an unpleasant mouthfeel and tends to adhere to the teeth. When the sticky mass is swallowed it then reacts further with gastric acid to form a carbonated raft of alginic acid which floats on the contents of the stomach and thereby suppresses gastric acid reflux. Therefore, there is a need in the art for a palatable, consumer acceptable solid dosage form, including a chewable tablet, of alginic acid and a bicarbonate salt.

### SUMMARY OF THE INVENTION

According to the present invention, there is a novel pharmaceutical composition of a chewable tablet which comprises alginic acid or a salt thereof, at least one water soluble carbonate radical precursor present in a proportion sufficient to form a metal alginic acid salt and carbonic acid upon contact with an aqueous solution or gastric fluid; at least one pharmaceutically acceptable calcium salt; and at least one of a first bulk sweetener or a binding agent. The calcium salt and the bulk sweetener or binding agent are combined together in a wet granulation process prior to admixture with the alginic acid. The formulation optionally has additional excipients, such as a second bulk sweetener, talc, mineral oil, an alkali metal salt of hexametaphosphate, a flavouring agent, an intense sweetener, or a dye.

Further according to the present invention, there is a pharmaceutical composition for a chewable tablet formed by a process comprising the following steps: providing an alginic acid or a salt thereof; providing a water-soluble carbonate radical precursor; providing a calcium salt; providing a first bulk sweetener; providing a binding agent; mixing the calcium salt and either or both of the bulk sweetener and the binding agent via wet granulation to form a mixture; and

blending the mixture with the alginic acid or salt thereof, the carbonate radical precursor, and with either the first bulk sweetener or the binding agent if not previously mixed with the calcium salt.

Further according to the present invention, there is a pharmaceutical composition fin the form of a chewable tablet. The composition has in admixture an alginic acid or a salt thereof; a water-soluble carbonate radical precursor; a calcium salt; a first bulk sweetener; and a binding agent.

Further according to the present invention, there is a pharmaceutical composition in powder form. The composition has in admixture an alginic acid or a salt thereof; a water-soluble carbonate radical precursor; a calcium salt; and a first bulk sweetener.

Further according to the present invention, there is a liquid pharmaceutical composition. The composition has in admixture an alginic acid or a salt thereof; a water-soluble carbonate radical precursor; a calcium salt; a first bulk sweetener; and water.

Further according to the present invention, there is a pharmaceutical composition for a chewable tablet. The composition has in admixture an alginic acid or a salt thereof; a water-soluble carbonate radical precursor; a calcium salt; a first bulk sweetener; and a binding agent. The calcium salt and either or both of said first bulk sweetener and said binding agent are blended via spray drying or direct compression prior to admixture with the alginic acid or salt thereof and the carbonate radical precursor.

### BRIEF DESCRIPTION OF THE DRAWINGS

FIG. 1 is a diagram of Rosett & Rice test results demonstrating the impact of varying excipients when used together on raft formation, and wherein AA is alginic acid.

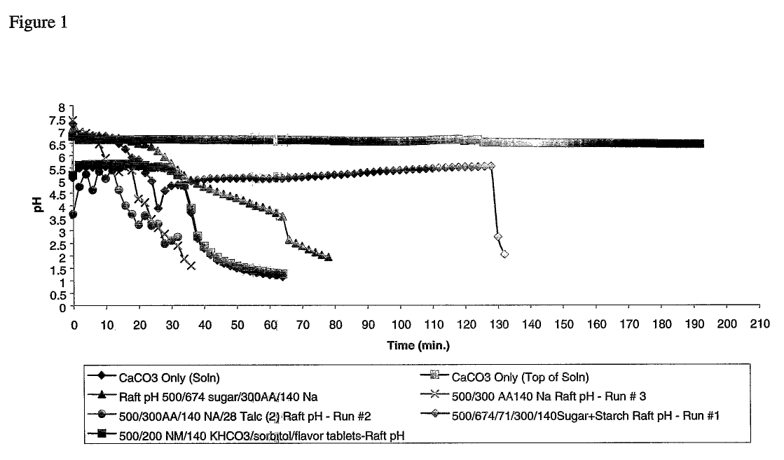

FIG. 2 demonstrates a schematic diagram of a Rosett & Rice test set up.

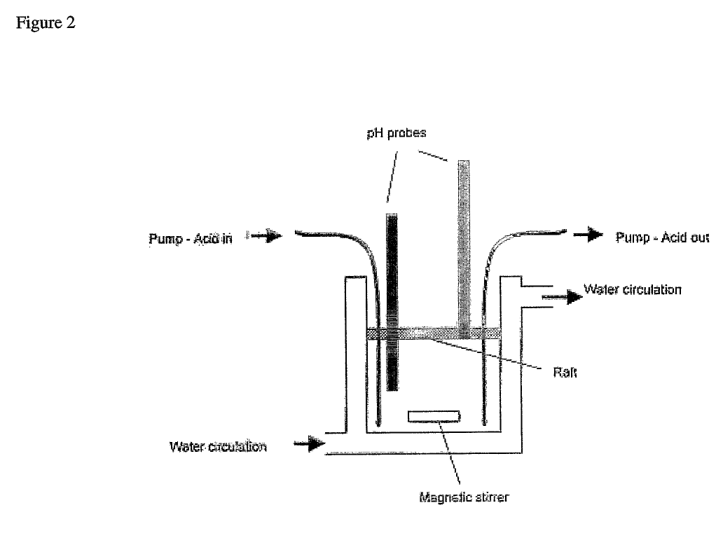

FIG. 3 is a diagram of Rosett & Rice test results (2 runs) demonstrating the impact of the addition of 140 mg of potassium bicarbonate on raft formation, along with 500 mg Calcium Carbonate granulation (no lubricant)+300 mg Alginic Acid, and 20 ml water.

FIG. 4 is a diagram of Rosett & Rice test results (2 runs) demonstrating the impact of the addition of 100 mg of sodium bicarbonate on raft formation along with the master lubricant blend +200 mg Alginic Acid.

FIG. 5 is a diagram of Rosett & Rice test results (2 runs) demonstrating the impact of the addition of 70 mg of sodium bicarbonate on raft formation along with the master lubricant blend +70 mg Sodium Bicarbonate and 20 ml water.

FIG. 6 is a diagram of Rosett & Rice test results (2 runs) demonstrating the impact of the addition of 140 mg of sodium bicarbonate on raft formation along with 500 mg Calcium Carbonate granulation (no lubricant)+300 mg Alginic Acid, and 20 ml water.

FIG. 7 is a diagram of Rosett & Rice test results (2 runs) demonstrating the impact of the addition of 70 mg of potassium bicarbonate and 70 mg of sodium bicarbonate on raft formation along with 500 mg Calcium Carbonate granulation (no lubricant)+300 mg Alginic Acid, 70 mg sodium bicarbonate, and 20 ml water.

FIG. 8 is a diagram of Rosett & Rice test results (2 runs) demonstrating the impact of the addition of 140 mg of sodium bicarbonate, master blend, 400 mg Alginic Acid, 500 mg Sorbitol and 20 ml water.

FIG. 9 is a diagram of Rosett & Rice test results (2 runs) demonstrating the impact of the addition of 140 mg of sodium bicarbonate, master blend, 300 mg Alginic Acid, 500 mg Sorbitol and 20 ml water.

FIG. 10 is a diagram of Rosett & Rice test results (2 runs) demonstrating the impact of the addition of 140 mg of sodium bicarbonate, master blend, 400 mg Alginic Acid, 500 mg Mannitol, and 20 ml water.

FIG. 11 demonstrates a study comparing the effect of the processed vs. unprocessed material, differing by their method of formation, i.e. Granulated or Processed vs. Dry blend. The graph describes the comparison of neutralization activity and raft performance of a Dry vs. Processed Blend with Starch.

FIG. 12 demonstrates a study comparing the effect of the processed vs. unprocessed material, differing by their method of formation, i.e. Granulated or Processed vs. Dry blend. The graph describes the comparison of neutralization activity and raft performance of a Dry vs. Processed Blend with Sugar.

FIG. 13 demonstrates a study comparing the effect of the processed vs. unprocessed material, differing by their method of formation, i.e. Granulated or Processed vs. Dry blend. The graph describes the comparison of neutralization activity and raft performance of a Dry vs. Processed Blend with Talc.

FIG. 14 demonstrates a study comparing the effect of the processed vs. unprocessed material, differing by their method of formation, i.e. Granulated or Processed vs. Dry blend. The graph describes the comparison of neutralization activity and raft performance of a Dry vs. Processed Blend with Sodium Hexametaphosphate.

FIG. 15 demonstrates a study comparing the effect of the processed vs. unprocessed material, differing by their method of formation, i.e. Granulated or Processed vs. Dry blend. The graph describes the comparison of neutralization activity and raft performance of a Dry vs. Processed Blend with Starch and Sugar.

FIG. 16 demonstrates a study comparing the effect of the processed vs. unprocessed material, differing by their method of formation, i.e. Granulated or Processed vs. Dry blend. The graph describes the comparison of neutralization activity and raft performance of a Dry vs. Processed Blend with Starch, Sugar and Talc.

FIG. 17 demonstrates a study comparing the effect of the processed vs. unprocessed material, differing by their method of formation, i.e. Granulated or Processed vs. Dry blend. The graph describes the comparison of neutralization activity and raft performance of a Dry vs. Processed Blend with Starch, Sugar, Talc, Light Mineral Oil, and Sodium Hexametaphosphate.

### DESCRIPTION OF THE INVENTION

The present invention is also directed to preparation of an alginic acid, or a salt thereof containing composition which comprises an effective amount of an antacid and which formulation is both palatable, and acceptable to the consumer, having improved organoleptic qualities. The resulting formulation will, in another embodiment, also provide a longer acting release of the antacid in the stomach.

Therefore, one embodiment of the present invention is a method for providing the continuous release of the antacid in the stomach to a mammal in need thereof, with an effective amount of a composition as defined herein.

The pharmaceutical composition, in another embodiment, will also provide and maintain over an extended period of time, the resulting raft/gel in the stomach contents. The composition provides for increased durability of the raft in the stomach contents, and in addition provides for maintenance of a reduced pH in the esophagus cavity. Therefore, another aspect of the present invention is a method of reducing gastric reflux, or prophylatic treatment of gastric reflux, in a mammal in need thereof, comprising administering to said mammal an effective amount of a composition as defined herein.

Another aspect of the present invention is a method of reducing heartburn symptoms, or prophylatic treatment of heartburn symptoms, in a mammal in need thereof, comprising administering to said mammal an effective amount of a composition as defined herein.

Another aspect of the invention is a method of reducing the incidence of gastric in the esophageal cavity in a human for a period of time, post ingestion of a meal sufficient to cause gastric reflux in said human for a time period of about 60 to about 480 minutes comprising administering to said human an effective amount of a composition as defined herein. Preferably, the time period is from about 120 to about 300 minutes or longer.

Another aspect of the present invention is a method of maintaining a pH of about 4.0 or higher in the esophageal cavity of a human in need thereof, for a time period of about 120 to about 300 minutes comprising administering to said human an effective amount of a composition as described herein. Preferably, the time period is from about 120 to about 180 minutes or longer. Also, the pH is preferably maintained at a pH of 5.0 or higher for this time period.

Another aspect of the present invention is a method of increasing the duration of a raft, greater than 30 minutes, in the stomach contents of a mammal by preparation of a wet granulate of calcium carbonate with a first bulk sweetener and/or a binding agent prior to admixture with alginic acid, or a salt thereof, and a water soluble carbonate radical precursor, such as sodium or potassium bicarbonate.

Another aspect of the present invention is a method of increasing the strength of a raft, greater than 30 minutes, in the stomach contents of a mammal by preparation of a wet granulate of calcium carbonate with a first bulk sweetener and/or a binding agent prior to admixture with alginic acid, or a salt thereof, and a water soluble carbonate radical precursor, such as sodium or potassium bicarbonate.

The present invention also provides for a composition which is readily compressible, durable for purposes of packaging and handling, and is disintegrable in a predictable manner such as by chewing, or if necessary by swallowing.

The pharmaceutical composition described herein, may also optionally comprise one or more pharmaceutically acceptable active agents or ingredients distributed within. A pharmaceutically acceptable active agent as defined herein follows the guidelines from the European Union Guide to Good Manufacturing Practice: Any substance or mixture of substances intended to be used in the manufacture of a drug (medicinal) product and that, when used in the production of a drug, becomes an active ingredient of the drug product. Such substances are intended to furnish pharmacological activity or other direct effect in the diagnosis, cure, mitigation, treatment, or prevention of disease or to affect the structure and function of the body.

Prophylaxis as defined herein shall mean, the tendency to prevent a substantial amount, <100%, of the disease or disorder for which the treatment is targetted.

The products of this invention are formulated such that a floating raft is formed on top of the gastric contents upon ingestion. In a physiological health disorder commonly referred to as heartburn or GERD (Gastroeosophageal reflux disease), the stomach acid is refluxed in to the esophagus, causing damage to the esophageal lining, hence the sensation of heartburn. A raft formed by the product of the present invention, will form a physical barrier to acid refluxing in to the esophagus, thereby preventing or reducing the continuous damage to the esophageal lining.

The raft is a matrix of alginate salts, the bulk of which is calcium, in co-existence with sodium or potassium ions. It is recognized that additional trace ions, such as magnesium may also be present as an impurity in one or more of the excipients. All of these trace ions may additionally enhance the raft formation, durability and strength thereof. The salt forms are a result of the interaction between the alginic acid and the salt source, such as calcium carbonate, sodium bicarbonate, and/or potassium bicarbonate. The resulting raft is made buoyant by the bicarbonate salt interacting with the stomach acid and generating carbon dioxide gas or bubbles. The bubbles are entrapped in the matrix and thus allow the raft to float on top of the gastric contents (the carbonated gel having a lower bulk density than the gastric acid). The raft so formed also entraps some of the unreacted calcium carbonate and provides a means for the antacid to continuously neutralise the gastric acid at the interface of the raft and the liquid below the raft. This mechanism provides a long lasting acid neutralization benefit. The calcium ions are believed to serve to cross-link the precipitated alginic acid molecules and thereby strengthen the gel matrix. The raft of this invention has been shown to last upwards of about 5 hours, or more, which otherwise would not possible. In a standard antacid formulation, the antacid component or ingredient neutralizes the acid immediately and does not have an extended neutralization effect. Immediate release antacids provide an acid neutralization benefit lasting upwards of about 40 minutes.

Thus, one of the features of the present invention is that the formulation provides for both an immediate as well as an extended neutralizing acid effect. While the active or therapeutic agent antacid entrapped in the floating raft matrix is providing the antacid effect, in this particular instance, a calcium salt, the resulting entity of the interaction with the stomach contents, i.e., calcium chloride, provides a source of calcium that is absorbed into the mammalians systemic circulation through the gastric mucosa and thereby also provides the health benefits of calcium. The extended release feature of this formulation, where calcium is released gradually over an extended period of time, is ideal for facilitating enhanced absorption of calcium. Thus another embodiment of the present invention is a method in the area of calcium supplementation for increasing the absorption of calcium in a mammal in need thereof, comprising administering to said mammal an effective amount of a composition as defined herein.

In addition, the present invention encompasses the discovery of an improved interaction between the excipients as formulated in this composition and the alginic acid and calcium carbonate, sodium or potassium bicarbonate. This improvement provides for formation of a much stronger raft than would be anticipated, as well as provides for an increased duration of the raft, i.e. a raft that lasts much longer on top of the stomach contents.

The interaction discovered here allows one to formulate the solid dosage form with a lower amount of alginic acid per tablet, such as 200 mg of alginic acid while unexpectently delivering the performance benefit that outlasts formulations containing 400 mg alginic acid per tablet. The raft pH is maintained at least two to four times longer and the raft strength is about 1.5 to 3 times stronger with formulations of the present invention. The lower, or reduced, use of alginic acid in a solid dosage form formulation, suitable for chewing in the mouth, provides not only considerable cost savings in raw material acquisition costs, but also provides for a more palatable taste and texture for the consumer.

An arbitrary criteria for use herein to assess the pH of the raft is one which should measure up to a pH of about 3.0 or above, and the duration of the raft is to last at least about two hours. The strength of the raft may vary but is preferably greater than about 3.5 grams, suitably greater than about 5.0, more suitably greater than about 6.0, and more suitably greater than 7.0. However, as the data will demonstrate herein, this is merely a baseline criteria and is not a limitation on the boundaries or scope of the invention herein. However, this baseline criteria has been used to understand what effects various excipients will produce on the raft formulation, duration and strength.

In the first embodiment of the invention the solid dosage form, such as a chewable tablet, comprises an antacid as the calcium salt, for example calcium carbonate, although any calcium salt meeting the required Food and Drug Administrations monograph for a calcium supplement or an antacid would be acceptable. Many of the pharmaceutically acceptable calcium salts meet these requirements, such as calcium citrate, calcium citrate maleate, calcium maleate, calcium lactate, calcium glyceryl phosphate, or calcium phosphate. The calcium must be adapted for compression into a tablet, and so may be preprocessed by any means suitable, such as slugging, roller compaction, aqueous wet granulation or non-aqueous wet granulation. A wide range of particle size, and grades of such directly compressible calcium are commercially available, and all are acceptable for use herein. To the now compressible calcium salt is added alginic acid, or a salt thereof, sodium or potassium bicarbonate (or a mixture thereof), and at least one excipient which contains one or more hydroxyl groups, such as a starch, a sugar, and/or a polyol, alone or in various combinations thereof. The tablet may also contain as necessary additional pharmaceutical excipients for manufacture of, stability of, disintegration of and customer appeal as necessary. These excipients may include additional sweeteners (conventional sweeteners, such as sucrose, dextrose, maltodextrin, sorbitol, or mannitol; or intense sweeteners, such as aspartame, sucralose, and/or acesulfamine K, etc., alone or in various combinations thereof), lubricants, flavors and colorants. The tablet may suitably be manufactured using conventional tabletting techniques.

Wet granulation is a method in which the active ingredient such as calcium carbonate is mixed with a binder and other excipients such as diluents, bulk sweeteners, disintegrants etc in a suitable granulator. A granulating solution such as water or a solution containing dissolved binder is added to the powder blend while mixing it thoroughly. This process allows the powder blend to become wet and agglomerate to form granules. These granules are then dried in a conventional tray drier or a fluid bed drier to obtain dry granules, which are then milled and screened to obtain granules with desirable particle size distribution. These granules are then mixed with additional ingredients such as diluents, bulk sweeteners, intense sweeteners, flavors, disintegrants, lubricants, anti-adherents, glidants etc., and compressed in to tablets.

Spray drying is another method to granulate powders to obtain spherical free flowing powders, which can be blended with various other excipients and compressed in to tablets. Typically in a spray drying operation, the active ingredient, binder and other desired excipients are suspended in water and sprayed using an atomizer in to the spray drier. The droplets so generated by the atomizer are dried to form granules, which can be screened and milled to obtain desired particle size.

Yet another method for manufacture of granules is a method called roller compaction, where dry blend of active ingredient(s), binder and other desired excipients are forced through a pair of rollers held under high pressure, where the powder compacts to form thin wafer like sheets, which are then milled and screened to obtain free flowing granules. Small amounts of water can be sprayed on to the powder blend prior to feeding in to the rollers, to enhance binding properties of ingredients in this process. The granules so obtained can be further processed to obtain tablets as explained above with the other processes.

Preferably, the calcium is produced as a granulate by any of the aforementioned granulation methods prior to admixture with the remaining excipients. More preferably the calcium carbonate is granulated with a first bulk sweetener, and/or a binding agent prior to admixture with the remaining excipients. More preferably, the granulate includes both the first bulk sweetener and the binding agent. For purposes herein, if the granulate includes both the first bulk sweetener and the binding agent, it may be referred to herein as a blend. Preferably, when the blend is a mixture of Calcium Carbonate, Confectionery Sugar, and Corn Starch and includes additional excipients, it is referred to as the master blend. The master blend will also include talc, mineral oil and sodium hexametaphosphate, unless otherwise indicated. In a preferred embodiment, the master blend comprises calcium carbonate in about 40% w/w; starch about 5%; confectioner's sugar about 50%; talc about 2%; light mineral oil about 1%; and sodium hexametaphosphate at about 0.4%.

The level of the calcium salt, such as calcium carbonate for use herein, is in the range of about 250 mg to about 1000 mg per tablet (free calcium), preferably about 250 mg to about 1000 mg per tablet, more preferably from about 250 mg to 750 mg, and most preferably about 500 mg/tablet. A useful but non-limiting range for the calcium salt is about 10% to about 50% by weight of the tablet.

The formulation may include cations in addition to the calcium from the calcium salt, such as in other antacids, including but not limited to magnesium carbonate, magnesium oxide, magnesium hydoxide, magnesium aluminate, aluminum hydroxide, or aluminum magnesium hydroxide, or combinations thereof. In an alternative embodiment the antacid is magnesium carbonate, or aluminum hyroxide, or combinations thereof. These antacids may be used alone, or in addition to the other antacids, and in amounts from about 5 to about 30% by weight of the tablet. Suitably, they are added from about 10-25, preferably about 20% by weight, or in a 100-250 mg/tablet dose, and suitably in a 200 mg dose per tablet.

Suitably for use herein is alginic acid. It is recognized that alginic acid salts such as calcium alginate, or sodium alginate, are also commercially available and may be used herein. One of the most useful properties of these water-soluble alginates is their ability to form viscous solutions at low concentrations. Because of the varied composition of the alginates, different alginates at the same concentration give solutions of differing viscosity. A level of alginic acid for use herein is in the range of about 140 to about 600 mg/tablet and most preferably about 200 mg/tablet. Other useful ranges include about 70 to about 600 mg/tablet, about 140 to about 300 mg, 200 to about 400 mg/tablet, and about 200 to about 300 mg/tablet. Experimental data indicates that there is no significant difference of pH profile among 200 mg, 250 mg, 300 mg and 400 mg of alginic acid and also pH profile of 200 mg alginic acid is more consistent than the other levels of alginic acid.

The water soluble carbonate radical precursor is a metal carbonate, or bicarbonate of an alkali or alkaline earth metal, such as the metals sodium, potassium, calcium, magnesium or manganese, and is present in an amount of about 50 mg to about 175 mg/tablet, preferably about 140 mg per tablet to 175 mg, more preferably about 110 to about 140 mg, respectively. Other useful ranges include 50 mg to about 200 mg/tablet and 70 mg to about 160 mg/tablet. Preferably, the water soluble carbonate radical is a salt of bicarbonate, and is suitably sodium or potassium bicarbonate, or a mixture thereof. Further in another embodiment, the water soluble carbonate radical precursor is a compound different than the calcium salt described above.

In an alternative embodiment of the present invention, it has been found that the water soluble carbonate radical or bicarbonate of an alkali or alkaline earth metal, can be replaced in whole or in part, with certain phosphate salts, such as sodium or potassium phosphate, or combinations thereof, in about a similar w/w % amount. The sodium, or potassium phosphate may be present in an amount of about 50 mg to about 175 mg/tablet, preferably about 140 mg per tablet to 175 mg, more preferably about 140 mg, respectively. Other useful ranges include 50 mg to about 200 mg/tablet and 70 mg to about 160 mg/tablet. It is recognized that if a portion of the water soluble carbonate radical or bicarbonate of an alkali or alkaline earth metal, is replaced by the sodium or potassium phosphate this may lead to various combinations of the actives being present.

The first bulk sweetener and the second bulk sweetener may be the same or different. The sweeteners may be conventional ones such as sugar, confectionery sugar, powdered sugar, sucrose, dextrose, glucose, lactose, fructose, or maltodextrin, or may be a polyol such as sorbitol, mannitol, xylitol, maltitol, fructose, polydextrose, erythritol, or combinations thereof.

Preferably the first bulk sweetener includes, but is not limited to a sugar which is dextrose, sucrose, fructose, lactose, confectionery sugar, powdered sugar, or is a polyol which is mannitol, sorbitol, xylitol, maltitol, maltose and polydextrose, or a mixture thereof. The first bulk sweetener is preferably sugar, mannitol, sucrose, or dextrose, or a combination thereof. More preferably it is confectionery sugar, powdered sugar or mannitol, as it appears to enhance raft strength and longevity.

The first bulk sweetener, if wet granulated with the calcium salt, is present in an amount from about 10% to about 30% of the tablet weight, preferably from about 15% to about 25% by weight

The amount of sugar in the master blend can therefore easily vary from half to double the amount as indicated. For instance, if the amount per tablet is about 655 mg, experimentation indicates that having this amount (327 mg) to doubling this amount (1300 mg) both produce a duration of raft in excess of 140 and 190 respectively, and raft strength of 10.68 and 11.05 respectively.

Preferably the second bulk sweetener is confectionery sugar, or powdered sugar, mannitol, sorbitol, sucrose, or dextrose, or a combination thereof. The second bulk sweetener if present, is in an amount from about 8% to about 50% of the tablet weight, preferably from about 10% to about 40% by weight. Another useful range is 8% to 40%, or 10 to 40% w/w.

The intense sweeteners may include, but not be limited to, aspartame, sucralose, acesulfamine K, and/or saccharin derivatives, or a mixture thereof. The intense sweetener is present in an amount from about 0.02% to about 0.12% of the tablet weight.

The bulk sweetener, such as mannitol, may alternatively be replaced in part with casein or gelatin, or combinations thereof on a w/w basis. For example if 400 mg mannitol were replaced equivalent amount of casein or gelatin, the raft duration has been found to be greater than 198 and 194 respectively, and a strength (force in g) of 10.30 and 8.19, respectively (where this is the time for the raft pH to reach 4.0, with a maximum time of 200 minutes).

If talc is present in the formulation, it is preferably in an amount up to about 1% of the tablet weight. Another useful range is about 0.5% to about 3% of the tablet weight.

If mineral oil is present in the formulation, it is in an amount up to about 1% of the tablet weight.

Suitable lubricants for use herein include, but are not limited to, magnesium stearate calcium stearate, sodium stearate, Cab-O-Sil (Colloidal Silicon Dioxide), Syloid™, stearic acid and talc. If a lubricant is present in the formulation, it is in an amount up to about 3% of the tablet weight. Colloidal Silicon Dioxide is also a synonym for fumed silica, light anhydrous silicic acid, silicic anhydride, and silicon dioxide fumed.

Suitable binding agents for use herein include, but are not limited to starches, polymers, natural gums, and low or medium viscosity cellulosic derivatives.

Suitably, if the binding agent is a starch, it is corn starch, modified corn starch, wheat starch, modified wheat starch, Starch 1500, or pregelatinized starch. Preferably the starch is corn starch or modified corn starch. The starch is present in an amount from about 1% to about 15% of the tablet weight. R&R testing has confirmed that in the Master blend, where approx. 72 mg of starch is present, doubling the dose (142 mg) and having the dose (36 mg), both produce a duration in minutes of the raft of greater than 194 minutes, and a strength (force in g.) of 10.90 and 12.36 respectively.

Suitably, when the binding agent is a low viscosity cellulosic derivative, it is a carbomer, hydroxypropylmethylcellulose (HPMC) including low to high viscosity versions thereof, hydroxypropylcellulose (HPC) including low to high viscosity versions thereof, microcrystalline cellulose (MCC), carboxymethylcellulose (CMC), hydroxyethylcellulose (HEC), or methylcellulose (MC); and combinations thereof. The cellulosic is present in an amount from about 1% to about 10% of the tablet weight. HPMC and HPC, both low viscosity has both been tested in the R&R assay herein, as has pectin, wheat starch and pregelatinized starch all meeting the defined criteria above. It should be noted that the determination of low, medium and high viscosity is based upon standard techniques and grading in the art. For instance a number following a grade of HPMC may indicate its approximate viscosity of a 2% solution at 20° C. For HPC, the commercial products are generally graded by their molecular weight, i.e. from 80,000 to about 1,150,000. These grades then exhibit viscosity results in mPas, ranging from 36-615, 410-740, etc. up to 2325-3300 mPas for instance (Klucel™, produced by Aqualon).

Suitably, when the binding agent is a natural gum it is pectin, gelatin, gum arabic, acacia, carrageenan, guar gum, or tragacanth. The gum is present in an amount from about 0.5% to about 7% of the tablet weight. Specifically, pectin has been found to have a duration of greater than 198 minutes, and a raft strength of 8.07.

Alternative binding agents also include povidone (PVP), polaxomer, polyethylene glycol (PEG), a polymethacrylate, or a combination thereof. It is recognized that the bulk sweeteners may also function as a binding agent, such as maltodextrin, mannitol, sorbitol, or polydextrose.

In addition the binders may include various polymers, similar to those already mentioned above, but also polyethylene oxide, sodium carboxymethylcellulose, polyvinyl alcohol, calcium polycarbophil, HPMC (medium viscosity), and polyethylene glycol (PEG), such as PEG 3350; or combinations thereof and/or combinations with other binding agents noted above. The polymers may be present in an amount from about 1 to 30% by weight, suitably from 5 to 25%, and more suitably about 20% w/w. Alternatively in mg amounts the polymers may be present in a 100-250 mg/tablet dose, suitably a 200 mg dose per tablet.

Suitably, if a dye or colorant or a flavorant is present in the formulation, it is present in conventional amounts.

In a typical tablet according to the invention, the metal carbonate or bicarbonate is used from about 2% to about 8% by weight of the tablet, and the calcium salt is used from about 10% to about 50% by weight of the tablet, the balance being active ingredients and any other formulation expedients desired. The binding agent if present is in an amount from about 1% to about 15%; the first bulk sweetener if present is in an amount from about 10% to about 30% and the second bulk sweetener if present is in an amount from about 10% to about 40% by weight of tablet.

A preferred embodiment of the present invention is the following composition:

| Ingredient Name | % w/w | mg/tablet or capsule |
|---|---|---|
| Master Blend | 51.7412 | 1293.53 |
| Alginic Acid | 8.0000 | 200.00 |
| Potassium Bicarbonate | 5.6000 | 140.00 |
| Mannitol | 32.5032 | 812.58 |
| Calcium Stearate | 0.4400 | 11.00 |
| Intense sweeteners | .0904 | 2.26 |
| dye | 0.1252 | 3.13 |
| Flavors | 1.5000 | 37.50 |
| | 100.0000 | 2500.00 |

In one aspect of the invention, the manufacturing of tablets herein involves a) granulation of the calcium carbonate; and b) dry blending the wet granulation of calcium carbonate with a first bulk sweetener, such as mannitol, and or a binding agent, such as starch, with alginic acid, potassium or sodium bicarbonate (or a mixture thereof); and optionally adding an intense sweetener, such as acesulfame K, and or sucralose, or a mixture thereof, flavors, a lubricating agent, such as calcium stearate, or magnesium stearate, talc and/or colloidal silicon dioxide; and then c) compressing the resulting blend using a tabletting machine into tablets.

In an alternative embodiment, the manufacturing of tablets herein involves a) wet granulation of the calcium carbonate with at least one of a first bulk sweeter and/or a binding agent; and b) dry blending the wet granulation of step (a) with a first bulk sweeter, if none was used in step (a) or a second bulk sweeter and a binding agent if one was not used in step (a) with alginic acid, potassium or sodium bicarbonate (or a mixture thereof); optionally to this blend may be added an intense sweetener, such as acesulfame K, and or sucralose, flavors, lubricants, such as calcium stearate, or magnesium stearate, talc and/or colloidal silicon dioxide; and then c) compressing the resulting blend using a tabletting machine into tablets.

In a preferred embodiment, the calcium carbonate is wet granulated with both a first bulk sweeter and a binding agent prior to admixing with the alginic acid, and potassium or sodium bicarbonate (or a mixture thereof). Preferably the first bulk sweetener is sugar NF, and the binding agent is corn starch NF. To the granulate is optionally added talc, light mineral oil, and sodium hexametaphosphate. This blend is then, preferably admixed with the alginic acid, the bicarbonate, a second bulk sweetener, such as mannitol, one or more intense sweeteners, flavours and lubricating agents.

Compositions of the present invention may be in product forms other than chewable tablets, such as a dry powder, and perhaps as a liquid. Depending on composition, the liquids may take the form of a suspension, dispersion, or emulsion. To form a liquid, ingredients are added to one or more solvents or vehicles, such as water, glycols, and the like.

The invention will now be described by reference to the following examples which are merely illustrative and are not to be construed as a limitation of the scope of the present invention.

Methodologies

In-vitro testing methodologies were set up in the laboratory based on the methodologies as shown below in order to determine the durability and strength of the resulting rafts. A measurement of a products performance pursuant to the criteria set forth herein, will produce high quality raft characteristics, such as wherein the pH of raft is about 3.0 or above, and the duration of the raft is at least two hours.

A. Rossett and Rice Tests:

The Rosett and Rice test is a continuous acid challenge test to model raft behaviour in vivo. The neutralization profile of the antacid, raft structure, raft appearance, duration the raft lasts, pH within the raft and pH of the liquid below the raft can be quantitatively and qualitatively measured, as appropriate.

The Rosett and Rice experiment is set up by using a 250 ml jacketed beaker connected to a constant temperature water bath equipped with a circulator. The water is circulated through the jacket at 37° C. (+/−3° C.) continuously through out the experiment. Two pH probes attached to two pH meters are used to measure the pH in the raft and below the raft. B They are calibrated, generally using a pH 7.00 and a pH 4.00 buffer. Both pH meters are connected to a computer by serial cables and the installed software collects the data and displays the pH values of both probes.

The contents of the beaker are stirred continuously using a magnetic stirrer at 100 RPM. The antacid sample to be tested is prewetted with 20 ml of water inside the jacketed beaker. A fixed volume of acid (100 ml) is added to the antacid slurry, which acid may be prewarmed. Various strengths of acid have been used in the R&R with 0.03N HCl considered the closest approximation of the physiological conditions of the stomach. The pH is monitored as further acid is added at a rate of 2 ml per minute. In this test a modification of the original test was used in which reactants are removed via a second pump to mimic gastric emptying. 0.1N HCl is used as the acid in our studies.

B. Texture Analysis:

To measure the strength of the raft a commercially available instrument, a Stable Micro Systems TAXT2I Texture Analyser was used. Two types of measurements can be made using this equipment, penetration measurements and pull through measurements. Since, penetration measurements can be made without disturbing the raft prior to measurements, this was the method of choice in the experiments herein. A modified Brookfield viscometer probe was used to measure the strength of the raft.

Texture analysis measurements were made on rafts formed using 0.1N HCl at 37 C at 5 min time point.

### Experiment 1

### Sodium and Potassium Bicarbonates

The effects of Sodium and Potassium Bicarbonates on the resulting raft at various levels has been evaluated within the context of the present invention. Two types of bicarbonates were selected as the best excipients to aid in development of the raft, sodium bicarbonate and potassium bicarbonate. Potassium Bicarbonate is preferred, as there are health benefits associated with potassium usage in contrast to sodium.

The Rossett & Rice and Texture Analyzer testing was used to evaluate various levels and reduce taste issues without compromising the raft formation, its lasting ability, and its strength. The table below outlines the level differences of the bicarbonates tested. In order not to introduce other variables into the formula the calcium salt, Calcium Carbonate was held constant at a level of 500 mg, and the Alginic Acid was held at a level of 300 mg. The tests were performed at a two-tablet/per dose level.

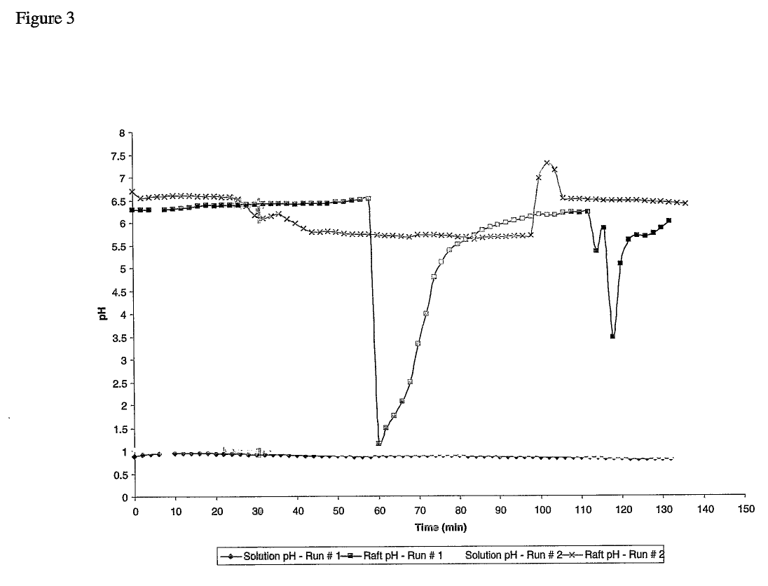

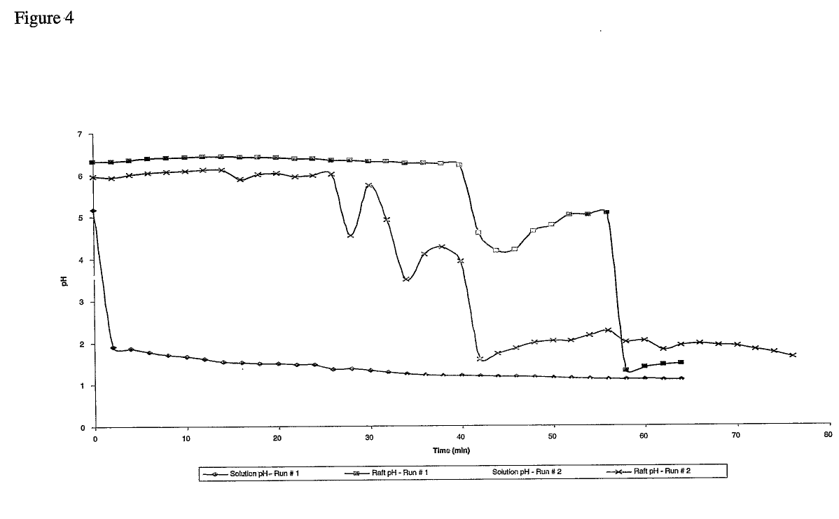

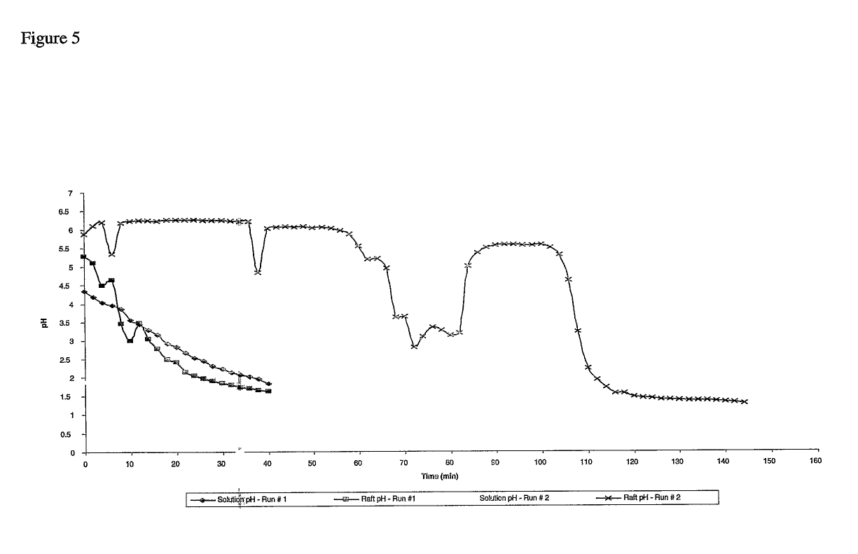

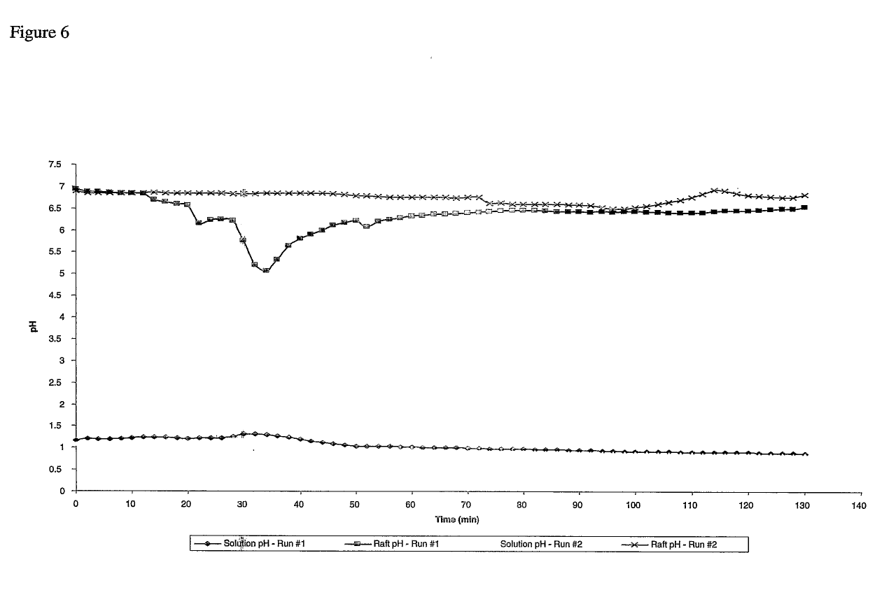

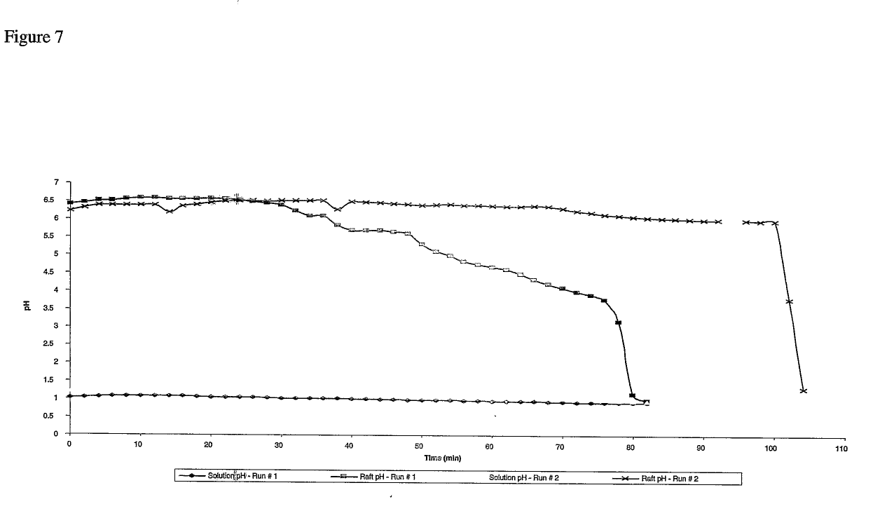

TABLE 1
BICARBONATE SAMPLES

| | Level Bicarbonate/ | | |
|---|---|---|---|
| Ingredient | tablet | | Results Reference |
| Sodium Bicarbonate | 140 | mg | FIG. 6 and Table 2 |
| Potassium Bicarbonate | 140 | mg | FIG. 3 and Table 2 |
| Sodium Bicarbonate | 100 | mg | FIG. 4 and Table 2 |
| Potassium Bicarbonate | 100 | mg | N/A* |
| Sodium Bicarbonate | 70 | mg | FIG. 5 and Table 2 |
| Potassium Bicarbonate | 70 | mg | N/A* |
| Sodium Bicarbonate/ | 70/70 | mg | FIG. 7 and Table 2 |
| Potassium Bicarbonate | | | |

*Results at 140 mg of Sodium vs. Potassium Bicarbonate provided similar data, therefore these samples are not shown herein, or were not tested.

B. Texture Analysis of samples were performed at a two-tablet dose. The samples in Table 2 are the average of two runs performed on the same experiment.

TABLE 2
TEXTURE ANALYSIS RESULTS

| Bicarbonate & Level | | Average Force (g.) |
|---|---|---|
| Sodium Bicarbonate | 140 mg | 5.451 |
| Potassium Bicarbonate | 140 mg | 16.706 |
| Sodium Bicarbonate | 100 mg | N/A* |
| Sodium Bicarbonate | 70 mg | N/A* |
| Sodium/Potassium Bicarbonate | 70 mg/70 mg | 10.719 |

*Samples were not tested due to Rossett & Rice results, reference FIGS. 4 and 5.

Conclusion:

Samples for Sodium Bicarbonate at levels of 140, 100, and 70 mg per tablet were tested. The Sodium Bicarbonate at the level of 140 mg per tablet provides acceptable raft results lasting for 130 minutes, while maintaining a pH between 5.5 and 6.0. Sodium Bicarbonate at 100 mg per tablet provided raft results lasting for 60 minutes, while maintaining a pH between a range of 6.5 and 4.0. The 70 mg per tablet sample provided results for pH between a range of 6.5 and 3.0 for approximately 100 minutes. It was determined that the 100 and 70 mg amounts as compared to the 140 mg fall short of the 2 hour time point chosen herein, with a pH above 3.0 for the better product performance, and herefore the 140 mg per tablet level for Sodium Bicarbonate was determined to be a more optimal level for use herein.

Rossett and Rice tested on at a level of 140 mg per tablet of Potassium Bicarbonate (FIG. 3*) and Sodium Bicarbonate (FIG. 6) provided consistent results of raft lasting for 130 minutes, while maintaining a pH between 5.5 and 6.0. The texture Analysis of these samples shows that the Potassium, with an average force (g.) of 16.706, provides a stronger raft structure than the Sodium, with an average force (g.) of 5.451. Based on this data from both of these excipients Potassium Bicarbonate at a level of 140 mg per tablet provides a stronger and long lasting raft (*note unusual dips in pH during testing are believed due to raft thickness reducing by the carbonate bubbles popping during the test).

In efforts to reduce negative taste effects of the Potassium Bicarbonate, both Potassium and Sodium were tested in combination at 70 mg per tablet equaling 140 mg total. The Rossett and Rice testing (FIG. 7) provided raft results lasting for approximately 90 minutes, while maintaining a pH between a range of 6.5 and 3.5. The 140 mg per tablet of either the Sodium or Potassium was the better choice.

It should be noted that the Rossett & Rice test, generally calls for 10 ml of water to be added to the powder sample to form slurry. It was discovered that 10 ml was not sufficient enough to wet the powder. A test sample was run following the method with the use of 20 ml to form the slurry. Results of the test sample using 20 ml compared to 10 ml showed a significantly better raft formation.

### Experiment 2

### Bulk Sweetener

In an effort to incorporate a bulk sweetener into the formulation, Rossett & Rice and Texture Analyzer testing was used to evaluate various representative sweeteners without compromising the raft characteristics and additionally improve the texture and test of the finished product.

The commonly used bulk sweeteners Dextrose, Sorbitol and Mannitol were selected for initial evaluation. Dextrose was performed at three different levels whereas Sorbitol and Mannitol were performed at only one level. The sweeteners were tested with the level of 400 mg Alginic Acid per tablet, except Sorbitol. Sorbitol was tested with the level of 300 mg and 400 mg per tablet. 20 ml of water was added to powder to form slurry for all of these experiments.

Rossett & Rice test and Texture analyzer test were performed for following experiments with one run for the Dextrose experiment and two runs for Sorbitol and Mannitol experiments, at a two-tablet dose level. The blend to which the second bulk sweetener was added consists of:

500 mg Calcium Carbonate+140 mg Sodium Bicarbonate+300 mg Alginic Acid+674 mg Confectioner's Sugar+71.43 mg Corn Starch+9.1 mg Sodium Hexametaphosphate. For purposes of this experiment this is referred to as the Master Blend.

a) Master Blend+Alginic acid 400 mg+Sodium Bicarbonate 140 mg+Dextrose 125 mg

b) Master Blend+Alginic acid 400 mg+Sodium Bicarbonate 140 mg+Dextrose 250 mg

c) Master Blend+Alginic acid 400 mg+Sodium Bicarbonate 140 mg+Dextrose 500 mg

d) Master Blend+Alginic acid 400 mg+Sodium Bicarbonate 140 mg+Sorbitol 500 mg

e) Master Blend+Alginic acid 300 mg+Sodium Bicarbonate 140 mg+Sorbitol 500 mg

f) Master Blend+Alginic acid 400 mg+Sodium Bicarbonate 140 mg+Mannitol 500 mg

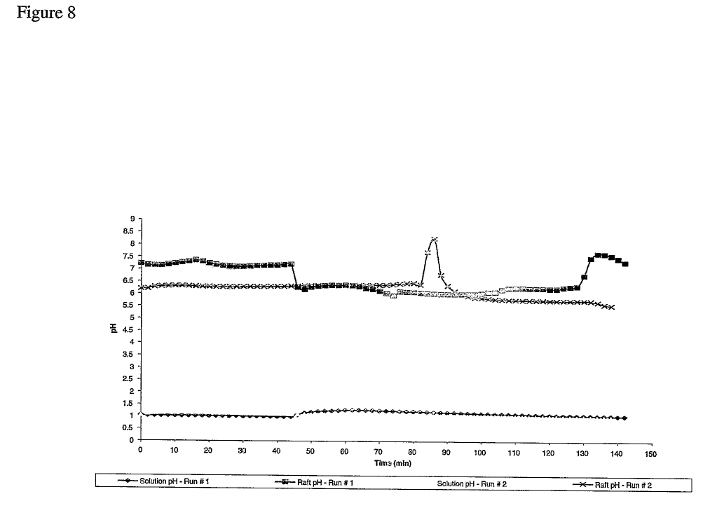

The experiment of Sorbitol 500 mg with 400 mg Alginic Acid and 140 mg Sodium Bicarbonate produced a strong raft. The solution below the raft had no floating particles compared to other experiments. The pH of the raft for Run #1 and Run #2 was measured above 5.5 until about 120 minutes. (See FIG. 8)

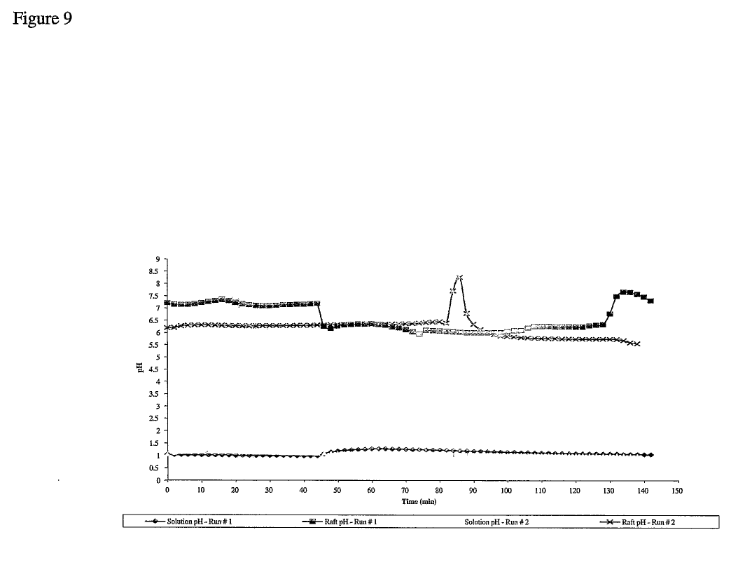

The experiment of Sorbitol 500 mg with 300 mg Alginic Acid and 140 mg Sodium Bicarbonate also produced a strong raft. The same observation like Sorbitol with 400 mg Alginic Acid was made for this experiment which was the solution below the raft had no floating particles compare to other experiments. The pH of the raft for Run #1 and Run #2 was measured above 6.0 until about 140 minutes. (See FIG. 9)

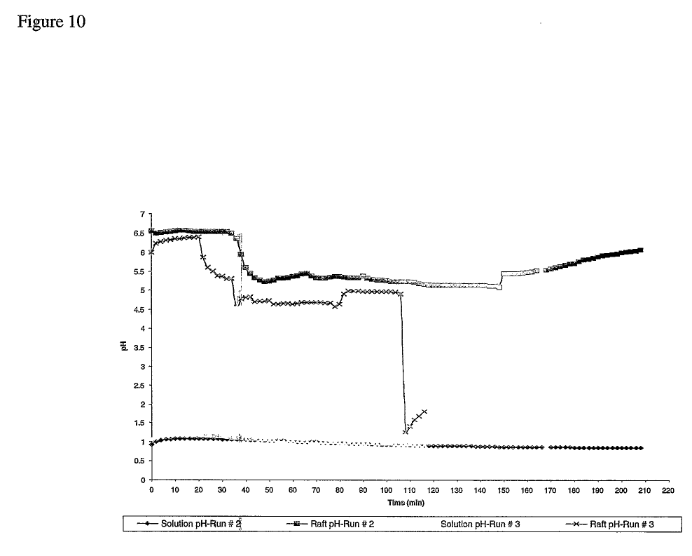

The experiment of Mannitol 500 mg with 400 mg Alginic Acid and 140 mg Sodium Bicarbonate also produced a strong raft. The pH of the raft for Run #1 and Run #2 was measured around 5.0 for about 100 minutes and 140 minutes, respectively. The unusual ups and downs in the raft pH are due to the raft thickness reducing by the bicarbonate bubbles popping during the test. (See FIG. 10).

Various samples featured in Table 3 below, were tested to determine the strength of the raft formed. The texture analysis of these samples shows that the Mannitol, with an average force (g.) of 12.332, provides a stronger raft structure than Sorbitol, with an average force (g.) of 9.862 and 6.629. Dextrose was not tested. Based on the Rosette & Rice and Texture Analysis data for both Mannitol and Sorbitol, either of the two raw materials are preferable for use a bulk sweetener. Sorbitol and Mannitol at a level of 500 mg per tablet produces a strong and long lasting raft, additionally sorbitol is more cost efficient than mannitol.

Texture Analysis of samples in Table 3 below is the average of two runs performed on the same experiment.

TABLE 3
TEXTURE ANALYSIS RESULTS

| Bulk Sweeteners | Average Force (g.) |
|---|---|
| Dextrose 125 mg + Alginic Acid 400 mg | N/A* |
| Dextrose 250 mg + Alginic Acid 400 mg | N/A* |
| Dextrose 500 mg + Alginic Acid 400 mg | N/A* |
| Sorbitol 500 mg + Alginic Acid 400 mg | 9.862 |
| Sorbitol 500 mg + Alginic Acid 300 mg | 6.629 |
| Mannitol 500 mg + Alginic Acid 400 mg | 12.332 |

*These samples were not performed due to their performance when conducting the Rossette and Rice testing.

### Experiment 3

### Components of Master Blend (MB)

Various component(s) of the MB have been separately tested in order to determine their role in the formation of strong raft in the presence of Alginic acid and Sodium Bicarbonate.

From previous experiments, it was decided to use 300 mg Alginic Acid per tablet and 140 mg Sodium Bicarbonate per tablet for all the experiments in this section. 20 ml. of water was added to the powder to form slurry for all the experiments. Rossett & Rice test and Texture analyzer test were performed for following experiments to meet the above objective. Two runs of each experiment, at a two-tablet dose, were performed for both the Rossett & Rice, and the Texture analyzer.
a) 500 mg Calcium Carbonate+140 mg Sodium Bicarbonate+300 mg Alginic Acid
b) 500 mg Calcium Carbonate+140 mg Sodium Bicarbonate+300 mg Alginic Acid+71.43 mg Corn Starch
c) 500 mg Calcium Carbonate+140 mg Sodium Bicarbonate+300 mg Alginic Acid+674 mg Confectioner's Sugar
d) 500 mg Calcium Carbonate+140 mg Sodium Bicarbonate+300 mg Alginic Acid+27.86 mg Talc
e) 500 mg Calcium Carbonate+140 mg Sodium Bicarbonate+300 mg Alginic Acid+4.55 mg Sodium Hexametaphosphate
f) 500 mg Calcium Carbonate+140 mg Sodium Bicarbonate+300 mg Alginic Acid+674 mg Confectioner's Sugar+71.43 mg Corn Starch
g) 500 mg Calcium Carbonate+140 mg Sodium Bicarbonate+300 mg Alginic Acid+674 mg Confectioner's Sugar+71.43 mg Corn Starch+4.55 mg Sodium Hexametaphosphate
h) 500 mg Calcium Carbonate+140 mg Sodium Bicarbonate+300 mg Alginic Acid+674 mg Confectioner's Sugar+71.43 mg Corn Starch+4.55 mg Sodium Hexametaphosphate in solution
i) 500 mg Calcium Carbonate+140 mg Sodium Bicarbonate+300 mg Alginic Acid+674 mg Confectioner's Sugar+71.43 mg Corn Starch+9.1 mg Sodium Hexametaphosphate
j) 500 mg Calcium Carbonate+140 mg Sodium Bicarbonate+300 mg Alginic Acid+674 mg Confectioner's Sugar+71.43 mg Corn Starch+9.1 mg Sodium Hexametaphosphate in solution

Texture Analysis of samples in Table 4 is the average of the two runs performed on the same experiment. Note: 300 mg Alginic Acid and 140 mg Sodium Bicarbonate were used in each run along with different components of Master Blend.

TABLE 4
TEXTURE ANALYSIS RESULTS

| | Average |
|---|---|
| Components | Force (g.) |
| Calcium Carbonate (alone) | 4.271 |
| Calcium Carbonate + Corn Starch | 4.011 |
| Calcium Carbonate + Confectioner's Sugar | 3.499 |
| Calcium Carbonate + Talc | 5.529 |
| Calcium Carbonate + 4.55 mg Sodium | 5.336 |
| Hexametaphosphate | |
| Calcium Carbonate + Confectioner's Sugar + | 3.798 |
| Corn Starch | |
| Calcium Carbonate + Confectioner's Sugar + | 4.711 |
| Corn Starch + 4.55 mg Sodium Hexametaphosphate | |
| Calcium Carbonate + Confectioner's Sugar + | 3.653 |
| Corn Starch + 9.1 mg Sodium Hexametaphosphate | |
| Calcium Carbonate + Confectioner's Sugar + | 4.017 |
| Cora Starch + 13.65 mg Sodium Hexametaphosphate | |

Various Observations:

Calcium Carbonate in presence of Alginic Acid and Sodium Bicarbonate did not form a strong raft as defined within the context of this invention. The raft was observed to be broken in a few pieces. The pH of raft was dropped below 3.0 within 15 to 30 minutes of run time.

Corn Starch in mixture with Calcium Carbonate and in presence of Alginic Acid and Sodium Bicarbonate did not help in forming strong raft. Loose particles were visible below the raft (in solution). The pH of raft was dropped below 3.0 within 15 to 30 minutes of run time.

Confectionery Sugar in mixture with Calcium Carbonate and in presence of Alginic Acid and Sodium Bicarbonate formed a weak raft. The pH of raft in Run #2 was dropped to 3.0 and below after about 65 minutes of run time where as in Run #2 the pH dropped to 3.0 and below after about 45 minutes of run time.

Talc in combination with Calcium Carbonate and in presence of Alginic Acid and Sodium Bicarbonate did not produce strong raft. The pH of the raft was below 3.0 after about 26 minutes.

Sodium Hexametaphosphate and Calcium Carbonate in presence of Alginic Acid and Sodium Bicarbonate did not produce strong raft. The pH of the raft, in Run #1 and Run #2, dropped below 3.0 after about 18 minutes and 55 minutes, respectively.

The mixture of Calcium Carbonate, Confectionery Sugar and Corn Starch in presence of Alginic Acid and Sodium Bicarbonate produced a thin and weak raft. The pH of the raft measured above 3.0 for 25 to 30 minutes.

The mixture of Calcium Carbonate, Confectionery Sugar, Sodium Hexametaphosphate (4.55 mg/tablet) and Corn Starch in presence of Alginic Acid and Sodium Bicarbonate produced a strong raft. The solution below the raft had much less particles floating as compared to other experiments. The pH of the raft for Run #1 and Run #2 was dropped to below 3.0 after about 90 minutes and 140 minutes, respectively.

The mixture of Calcium Carbonate, Confectionery Sugar, Corn Starch, and Sodium Hexametaphosphate (4.55 mg/tablet) in solution with presence of Alginic Acid and Sodium Bicarbonate produced a reasonably strong raft. The pH of the raft for Run #1 and Run #2 was dropped to below 3.0 after about 130 minutes and 120 minutes, respectively.

The mixture of Calcium Carbonate, Confectionery Sugar, Sodium Hexametaphosphate (9.1 mg/tablet) and Corn Starch in presence of Alginic Acid and Sodium Bicarbonate produced a raft like a gel or sponge. The pH of the raft, in Run #1 dropped below 3.0 after about 186 minutes while in Run #2 after about 90 minutes.

In brief, the mixture of Calcium Carbonate, Confectionery Sugar, Sodium Hexametaphosphate (4.55 mg/tablet) and Corn Starch in presence of Alginic Acid and Sodium Bicarbonate produced a long lasting raft. Moreover, the double amount (9.1 mg/tablet) of Sodium Hexametaphosphate did not add any additional advantage for the formation of strong raft. Furthermore, adding Sodium Hexametaphosphate in powder form, or in solution, did not create any effect in the formation of raft. Therefore, if Sodium Hexametaphosphate is desired to be added, it can be in either form during the processing of the formulation.

For the texture analysis, various samples were tested to determine the strength of the raft formed. The texture analysis of these samples shows that the mixture of Calcium Carbonate and Talc, with an average force (g.) of 5.529, the mixture of Calcium Carbonate and Sodium Hexamethaphosphate, with an average force (g) of 5.336 and the mixture of Calcium Carbonate, Confectioner's Sugar, Corn Starch, 4.55 mg Sodium Hexametaphosphate, with an average force (g) of 4.711 provides a better texture than other samples.

Based on the Rosette & Rice and Texture Analysis data, it was concluded that all the raw materials (Calcium Carbonate, Confectioner's Sugar, Corn Starch, 4.55 mg Sodium Hexametaphosphate) together form a strong raft as well as provide good texture.

### Experiment 4

### Formation of a Raft with a Dry Blend of CaCO₃

In order to determine the effects of a dry blend of calcium carbonate the ingredients in the table below were all weighed out individually. They were combined in ajar and mixed thoroughly by tumbling the glass jar. To remove clumps, the blend was passed through #20 sieve mesh screen

Formula 1: Dry Blend with CaCO₃

| Ingredient | mg/tab | |
|---|---|---|
| Calcium Carbonate | 500 | mg/tab |
| Alginic Acid F120 NM | 200 | mg/tab |
| Potassium Bicarbonate | 140 | mg/tab |
| Mannitol 200 SD | 809.48 | mg/tab |
| Calcium Stearate NF | 11 | mg/tab |
| Acesulfame K | 1.12 | mg/tab |
| Sucralose, NF | 1.12 | mg/tab |
| Flavours | 27.4 | mg/tab |

In a similar manner a formulation of a Dry Blend without CaCO₃ was produced, having the following formula:

Formula 2: Dry Blend without CaCO₃

| Ingredient | mg/tab | |
|---|---|---|
| Alginic Acid F120 NM | 200 | mg/tab |
| Potassium Bicarbonate | 140 | mg/tab |
| Mannitol 200 SD | 809.48 | mg/tab |
| Calcium Stearate NF | 11 | mg/tab |
| Acesulfame K | 1.12 | mg/tab |
| Sucralose, NF | 1.12 | mg/tab |
| Flavours | 27.4 | mg/tab |

Formation of Granulated Blends

The components were combined in respective jars labelled A-I;

Distilled water was added to each jar to prepare granulated materials for testing;

The granulations were dried overnight, then ground with a mortar and pestle.

TABLE 5
Formation of Granulated Blends

| | | | Theoretical | Actual |
|---|---|---|---|---|
| | | | Wt.(g)/ | Wt.(g)/ |
| | Excipients | mg/tab. | 20 tab. | 20 tab. |
| A | Calcium Carbonate | 500.00 | 10.0000 | 10.0471 |
| | Starch(Corn) | 71.43 | 1.4286 | 1.4224 |
| B | Calcium Carbonate | 500.00 | 10.0000 | 10.0259 |
| | Sugar, Powder | 654.79 | 13.0958 | 13.097 |
| C | Calcium Carbonate | 500.00 | 10.0000 | 10.0023 |
| | Talc | 27.68 | 0.5536 | 0.5534 |
| D | Calcium Carbonate | 500.00 | 10.0000 | 10.0091 |
| | Light Mineral Oil | 15.08 | 0.3016 | 0.3015 |
| E | Calcium Carbonate | 500.00 | 10.0000 | 10.0471 |
| | Sod. Hexa Metaphosphate | 4.55 | 0.091 | 0.0913 |
| F | Calcium Carbonate | 500.00 | 10.0000 | 10.0163 |
| | Starch(Corn) | 71.43 | 1.4286 | 1.4269 |
| | Sugar, Powder | 654.79 | 13.0958 | 13.0905 |
| G | Calcium Carbonate | 500.00 | 10.0000 | 10.003 |
| | Starch(Corn) | 71.43 | 1.4286 | 1.4268 |
| | Sugar, Powder | 654.79 | 13.0958 | 13.0931 |
| | Talc | 27.68 | 0.5536 | 0.555 |
| H | Calcium Carbonate | 500.00 | 10.0000 | 10 |
| | Starch(Corn) | 71.43 | 1.4286 | 1.4262 |
| | Sugar, Powder | 654.79 | 13.0958 | 13.105 |
| | Talc | 27.68 | 0.5536 | 0.5546 |
| | Light Mineral Oil | 15.08 | 0.3016 | 0.3081 |
| I | Calcium Carbonate | 500.00 | 10.0000 | 10.009 |
| | Starch(Corn) | 71.43 | 1.4286 | 1.4251 |
| | Sugar, Powder | 654.79 | 13.0958 | 13.08 |
| | Talc | 27.68 | 0.5536 | 0.5591 |
| | Light Mineral Oil | 15.08 | 0.3016 | 0.3051 |
| | Sod. Hexa Metaphosphate | 4.55 | 0.091 | 0.0938 |

Results:
Rossett & Rice Tests

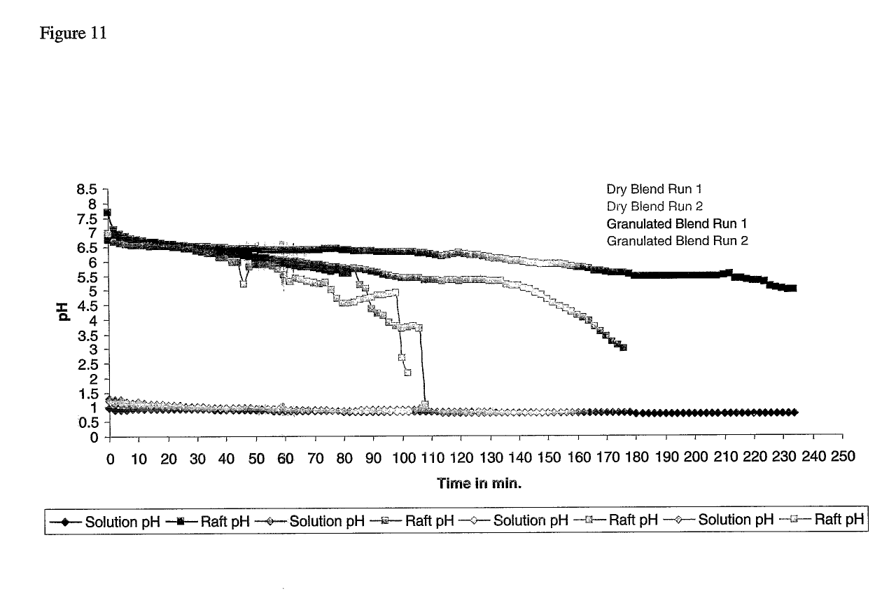

FIG. 11 demonstrates a study comparing the effect of the processed vs. unprocessed material. The blends are similar in composition, but differ in method of formation, i.e. Granulated vs. dry blend.
Unprocessed/Dry Blend: Formula 1+Starch
Processed/Granulated Blend: Formula 2+CaCO₃ with Starch

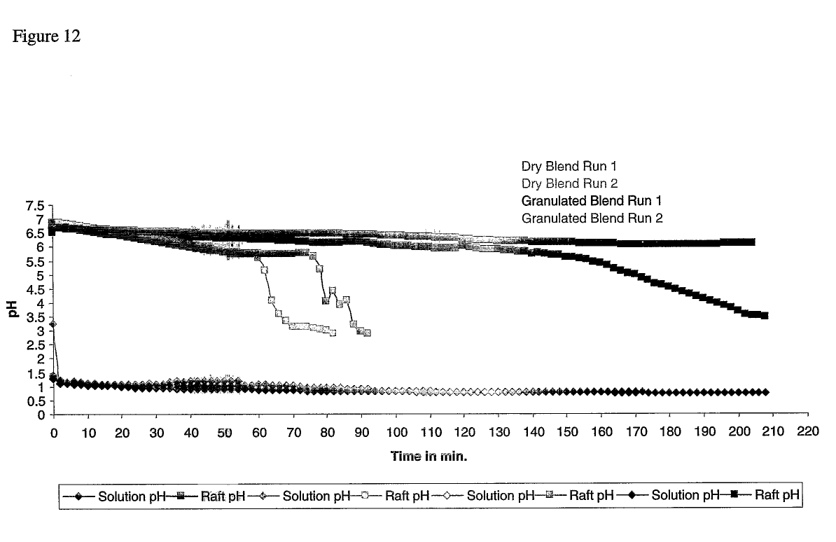

FIG. 12 demonstrates a study comparing the effect of processed vs. unprocessed material. The blends are similar in composition, but differ in method of formation, i.e. Granulated vs. dry blend.
Unprocessed/Dry Blend: Formula 1+Sugar
Processed/Granulated Blend: Formula 2+CaCO₃ with Sugar

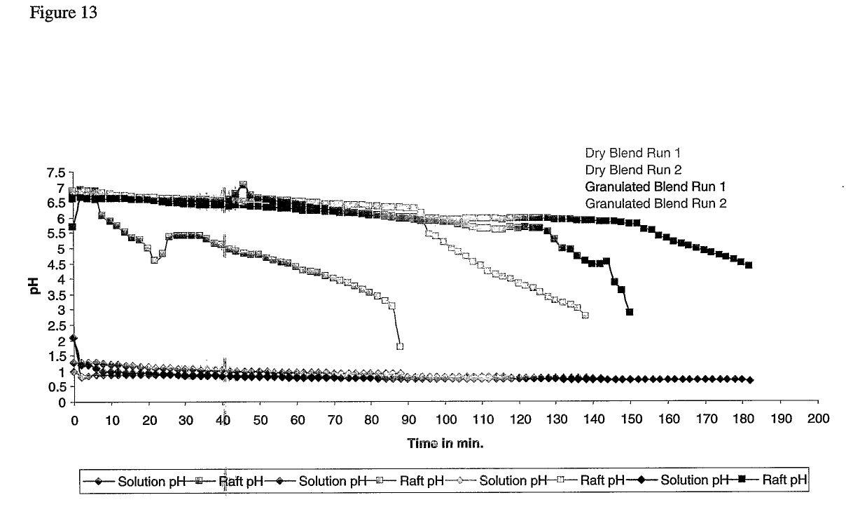

FIG. 13 demonstrates a study comparing the effect of processed vs. unprocessed material. The blends are similar in composition, but differ in method of formation, i.e. Granulated vs. dry blend.
Unprocessed/Dry Blend: Formula 1+Talc
Processed/Granulated Blend: Formula 2+CaCO₃ with Talc

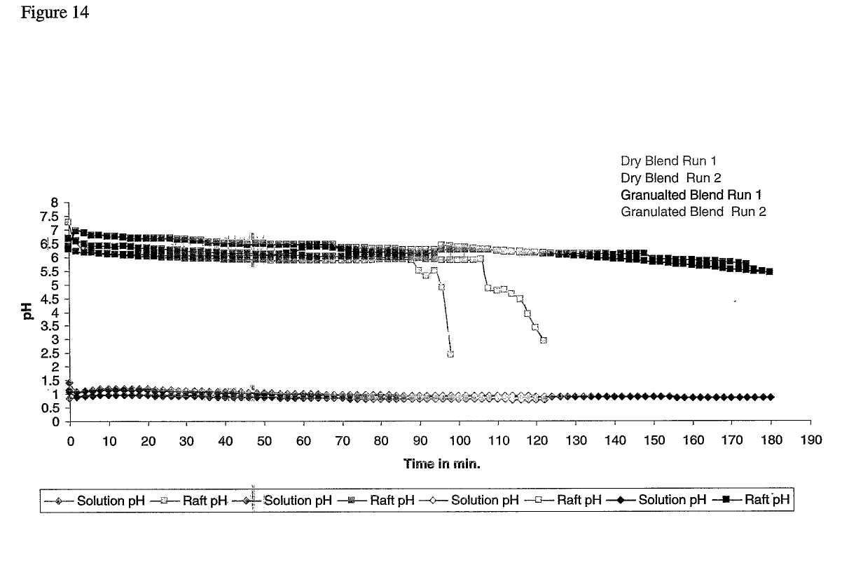

FIG. 14 demonstrates a study comparing the effect of processed vs. unprocessed material. The blends are similar in composition, but differ in method of formation, i.e. Granulated vs. dry blend.
Unprocessed/Dry Blend: Formula 1+NaHMP
Processed/Granulated Blend: Formula 2+CaCO₃ with NaHMP

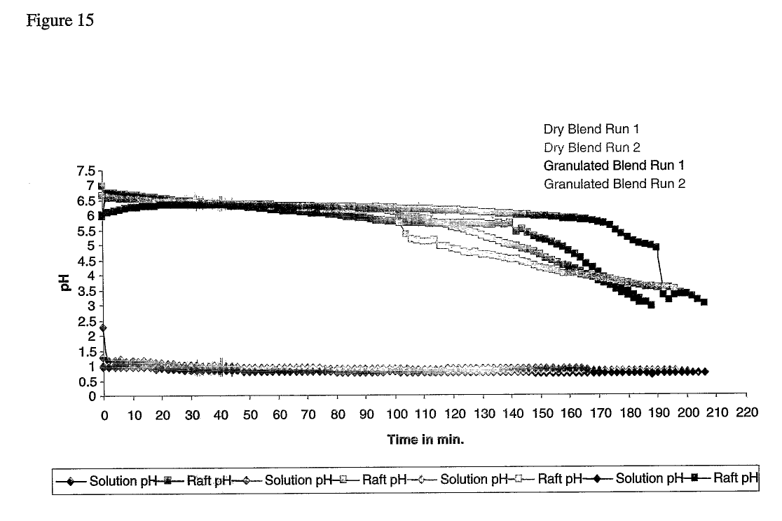

FIG. 15 demonstrate a study comparing the effect of processed vs. unprocessed material. The blends are similar in composition, but differ in method of formation, i.e. Granulated vs. dry blend.
Unprocessed/Dry Blend: Formula 1+Starch+Sugar
Processed/Granulated Blend: Formula 2+CaCO₃ with Starch and Sugar

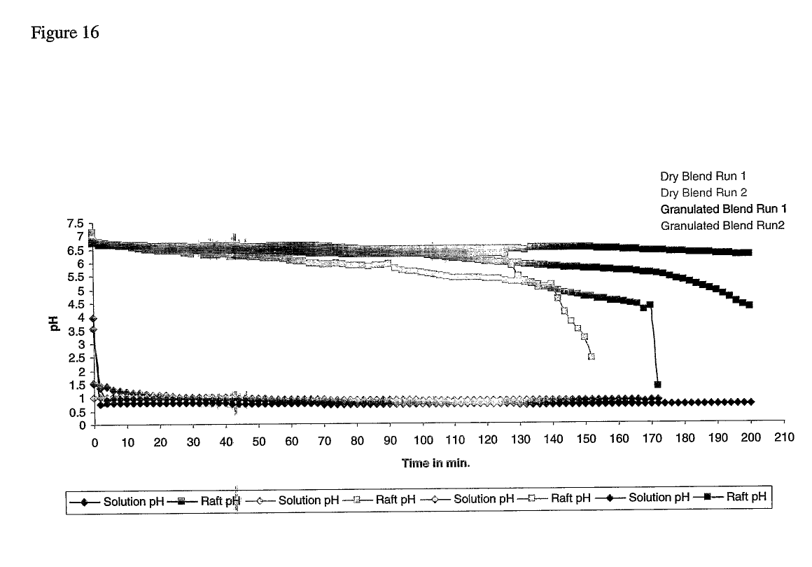

FIG. 16 demonstrates a study comparing the effect of processed vs. unprocessed material. The blends are similar in composition, but differ in method of formation, i.e. Granulated vs. dry blend.
Unprocessed/Dry Blend: Formula 1+Starch+Sugar+Talc
Processed/Granulated Blend: Formula 2+CaCO3 with Starch, Sugar and Talc

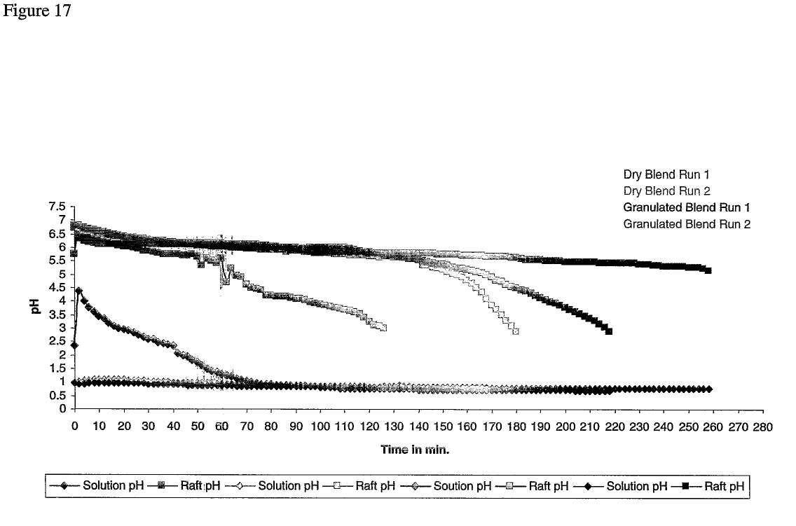

FIG. 17 demonstrates a study comparing the effect of processed vs. unprocessed material. The blends are similar in composition, but differ in method of formation, i.e. Granulated vs. dry blend.
Unprocessed/Dry Blend: Formula 1+Starch+Sugar+Talc+LMO+NaHMP
Processed/Granulated Blend: Formula 2+CaCO₃ with Starch, Sugar, Talc, LMO, and NaHMP
   Analysis of Rossett & Rice Tests

Table 6, shown below, demonstrates the analysis of the Blends as described above.

| Sample ID | Last Reading in mini (Last pH) |
|---|---|
| Dry Blend: A | |
| Run One | 108 (1.05) |
| Run Two | 102 (2.15) |
| Granulated Blend: A | |
| Run One | 234 (4.96) |
| Run Two | 176 (2.97) |
| Dry Blend: B | |
| Run Two | 82 (2.89) |
| Run Three | 92 (2.88) |
| Granulated Blend: B | |
| Run One | 208 (3.46) |
| Run Two | 204 (6.11) |
| Dry Blend: C | |
| Run One | 88 (1.78) |
| Run Two | 138 (2.74) |
| Granulated Blend: C | |
| Run One | 150 (2.85) |
| Run Two | 182 (4.39) |
| Dry Blend: D | |
| Run One | 44 (2.11) |
| Run Two | 122 (3.04) |
| Granulated Blend: D | |
| Run One | 172 (5.34) |
| Run Two | 180 (3.03) |
| Dry Blend: E | |
| Run One | 122 (2.92) |
| Run Two | 98 (2.43) |
| Granulated Blend: E | |
| Run One | 180 (5.41) |
| Run Two | 178 (5.45) |
| Dry Blend: F | |
| Run One | 184 (3.03) |
| Run Two | 200 (3.33) |
| Granulated Blend: F | |
| Run One | 188 (2.91) |
| Run Two | 206 (2.99) |
| Dry Blend: G | |
| Run One | 172 (1.33) |
| Run Two | 152 (2.40) |
| Granulated Blend: G | |
| Run One | 200 (4.24) |
| Run Two | 200 (6.19) |
| Dry Blend: H | |
| Run One | 104 (1.86) |
| Run Two | 132 (2.86) |
| Granulated Blend: H | |
| Run One | 172 (2.98) |
| Run Two | 176 (5.85) |
| Dry Blend: I | |
| Run One | 126 (3.01) |
| Run Two | 180 (2.88) |
| Granulated Blend: I | |
| Run One | 218 (2.89) |
| Run Two | 258 (5.16) |

Texture Analyzer

The Table below provides the Texture Analyzer results of raft strength measured in grams, See reference blends below.

Reference Blends

Unprocessed Blends/Dry Blends:

A. Formula 1+Starch
B. Formula 1+Sugar
C. Formula 1+Talc
D. Formula 1+Light mineral oil
E. Formula 1+Sodium Hexametaphosphate.
F. Formula 1+Starch and Sugar
G. Formula 1+Starch and Sugar and Talc
H. Formula 1+Starch and Sugar and Talc and LMO
I. Formula 1+Starch, Sugar, Talc, LMO, Sodium HMP

Processed Blends/Granulated Blends: (formula 2 is the formation of a dry blend without CaCO3 as described above
A. Calcium Carbonate (500 mg)+Starch (granulate and dry) and Formula 2
B. Calcium Carbonate (500 mg)+Sugar (granulate and dry) and Formula 2
C. Calcium Carbonate (500 mg)+Talc (granulate and dry) and Formula 2
D. Calcium Carbonate (500 mg)+LMO (granulate and dry) and Formula 2
E. Calcium Carbonate (500 mg)+Sodium HMP (granulate and dry) and Formula 2
F. Calcium Carbonate (500 mg)+Starch and Sugar (granulate and dry) and Formula 2
G. Calcium Carbonate (500 mg)+Starch, Sugar, Talc (granulate and dry) and Formula 2
H. Calcium Carbonate (500 mg)+Starch, Sugar, Talc, LMO (granulate and dry) and Formula 2

I. Calcium Carbonate (500 mg)+Starch, Sugar, Talc, LMO, Sodium HMP (granulate and dry) and Formula 2

TABLE 7
Average forces of the Texture Analyzer Tests

| Sample Blend | Average force in grams |
|---|---|
| A: Dry Blend | 4.271 |
| A: Granulated Blend | 5.402 |
| B: Dry Blend | 4.654 |
| B: Granulated Blend | 7.354 |
| C: Dry Blend | 5.709 |
| C: Granulated Blend | 5.114 |
| D: Dry Blend | 5.172 |
| D: Granulated Blend | 4.047 |
| E: Dry Blend | 5.038 |
| E: Granulated Blend | 5.367 |
| F: Dry Blend | 5.435 |
| F: Granulated Blend | 5.788 |
| G: Dry Blend | 6.488 |
| G: Granulated Blend | 7.43 |
| H: Dry Blend | 5.767 |
| H: Granulated Blend | 9.015 |
| I: Dry Blend | 4.893 |
| I: Granulated Blend | 11.016 |

Rossett & Rice Tests

The five excipients starch, sugar, talc, light mineral oil, and sodium hexametaphosphate were each tested in various blends for their effect on raft formation when processed or not processed.

The results are as follows:

Blends with Starch

a. Unprocessed/Dry Blend: Both run one and run two depict a weak raft that lasts for approximately 100 minutes.
b. Processed/Granulated Blend: Although there is over an hour discrepancy between the two runs, it is evident that processing has a significant effect on the raft with starch.
   Blends with Sugar
a. Unprocessed/Dry Blend: Both runs one and two last for less than 100 minutes, the raft is weak.
b. Processed/Granulated Blend: Both runs demonstrate the ability of granulated sugar with a blend to last for at least 200 minutes, a dramatic improvement from previous runs with sugar.
   Blends with Talc
a. Unprocessed/Dry Blend: Raft not evaluated.
b. Processed/Granulated Blend:Raft not evaluated.
   Blends with LMO
a. Unprocessed/Dry Blend: The raft may not be stable since results of two runs differ by over an hour.
b. Processed/Granulated Blend: Results from first two runs of unprocessed material are very varied, but most likely processing has no effect on LMO.
   Blends with NaHMP
a. Unprocessed/Dry Blend: Run two lasts for only 24 minutes longer than the first, so the results are consistent. Also, the runs terminate at 98 minutes and 122 minutes, therefore the raft is relatively stronger than blends with other excipients.
b. Processed/Granulated Blend: The consistency and durability of the unprocessed blends are surpassed by the results of the processed blends, therefore it can be concluded that granulation has an effect on sodium hexametaphosphate.
   Blends with Starch and Sugar
a. Unprocessed/Dry Blend: The combination of starch and sugar alone has a strong effect on the raft neutralization activity.
b. Processed/Granulated Blend: Although processing does not have an effect on the combination of these two excipients, the durability of the raft is clearly controlled by the composition of the raft rather than the formation of it.
   Blends with Starch, Sugar and Talc
a. Unprocessed/Dry Blend: Without processing, one can conclude that the combination of these three excipients has a strong impact on raft neutralization activity
b. Processed/Granulated Blend: Although it is difficult to see in the figure, processing does have a slight impact on the excipient combination. Even after 200 minutes, the pH is still relatively neutral.
   Blends with Sugar, Starch, Talc and LMO
a. Unprocessed/Dry Blend: Without processing, the combination of these four excipients forms a raft weaker than expected.
b. Processed/Granulated Blend: In the figure with the four excipients, it is apparent that processing improved the raft neutralization activity
   Blends with Starch, Sugar, Talc, LMO, and NaHMP
a. Unprocessed/Dry Blend: The unprocessed material lasts for a very long time, indicating that the combination of excipients is effective
b. Processed/Granulated Blend: Although the two runs are slightly different, it is evident that processing had an impact on the excipients. This can be concluded because both runs ran well over 200 minutes.
   Texture Analyzer
The texture analyzer is designed to measure the strength of the raft. It is important for the raft to have a high penetration force to be able to protect against acid reflux. In the results, as seen in above, processed blends were consistently resulting in higher penetration forces than dry blends. The only exceptions were LMO and Talc because of their poor solubility in water (solubility in water is essential because of the nature of the granulation process). Therefore, it can be concluded that processing has a greater effect on raft strength than without processing.

When analyzing the excipients as individual blends, the results show that sugar alone, once processed, provides one of the strongest rafts. However, once the ingredients of the MB are combined, all five excipients, starch, sugar, talc, light mineral oil, and sodium hexametaphosphate, when combined produce the strongest raft formed.

Thus this experiment demonstrates the blends processed by way of a granulation are more likely to result in longer lasting and stronger raft formations than an unprocessed blend.

### Experiment 5

### Excipient Variations

The following tables demonstrate using the methodologies described herein, more fully the differences in raft strength between a non-compressed calcium (Table 8), a granulate, processed blend (Table 9), and a granulated, processed blend which has both a sweetener and a binding agent granulated together (Table 10). In Table 10, reference is made to the MB formulation, which is described earlier.

For the on processed calcium carbonate powder, the brand name “Albagloss” made by a company called Specialty Minerals was used. This material is 100% pure calcium carbonate and is not a granulation.

For the experiments of Table 8, the unprocessed blends, the excipients have been mixed as listed in the Table which the R&R and Texture Analysis conducted thereon. The experiments with the unprocessed blends did not involve any granulation or tabletting.

In case of the processed blends (i.e., granulated blends) the following preparation steps were taken. The calcium carbonate used in these blends is same as the one used in the unprocessed blends (Albagloss). A double amount (two tablets dose equivalent) of CaCO₃ and other excipients shown in the table were weighed out and mixed and ground together in a coffee grinder, except alginic acid, KHCO₃ and mannitol. A suitable amount of water was added to form good granules. The granules were then dried in an oven and milled using mortar and pestle to obtain desired particle size. The granules were then blended with alginic acid, potassium bicarbonate and mannitol, and the resulting final blend was used in R&R and Texture analysis testing.

The mixture is transferred to a jacketed 250 ml. Beaker. To the beaker was added 20 ml. of 37° C. water and mixed well until dispersed completely. To this is added 100 ml of 37° C. 0.1N HCl to form a raft. A magnetic stirrer is added to the jacketed 250 ml. beaker and started to rotate at speed of 100 rpm. To this is inserted two rubber tubes into the testing beaker, into which is pumped at 37° C. 0.1N HCl solution and pump out 37° C. 0.1N HCl solution at the speed of 2 ml/minute concurrently. One pH probe is inserted to measure the pH of solution below the raft and another pH probe inserted to measure pH of the raft. The pH is measured at 2 minutes interval and both pH measurements are recorded. An average length of two tests in minutes to reach pH 4.0 is tabulated in the tables shown below.

TABLE 8
Raft Data

| Simple Unprocessed Physical Blend | | | | | | | | | Raft | R&R test |
|---|---|---|---|---|---|---|---|---|---|---|
| | Alginic | | | | | | | | Strength | Raft in min. |
| CaCO₃ | acid | KHCO₃ | Mannitol | Starch | Sugar | Talc | LMO | NaHMP | Force in | to reach |
| 500 mg | 200 mg | 140 mg | 800 mg | 71.43 mg | 654.8 mg | 27.68 mg | 15.08 mg | 4.55 mg | gram | to pH 4.0 |
| CaCO₃ | Alg-acid | KHCO₃ | | | | | | | 4.505 | 12 |
| CaCO₃ | Alg-acid | KHCO₃ | Mannitol | | | | | | 6.077 | 31 |
| CaCO₃ | Alg-acid | KHCO₃ | Mannitol | Starch | | | | | 4.271 | 97 |
| CaCO₃ | Alg-acid | KHCO₃ | Mannitol | | Sugar | | | | 4.654 | 73 |
| CaCO₃ | Alg-acid | KHCO₃ | Mannitol | | | Talc | | | 5.709 | 93 |
| CaCO₃ | Alg-acid | KHCO₃ | Mannitol | | | | LMO | | 5.172 | 80 |
| CaCO₃ | Alg-acid | KHCO₃ | Mannitol | | | | | NaHMP | 5.038 | 106 |
| CaCO₃ | Alg-acid | KHCO₃ | Mannitol | Starch | Sugar | | | | 5.435 | 163 |
| CaCO₃ | Alg-acid | KHCO₃ | Mannitol | Starch | Sugar | Talc | | | 6.488 | 157 |
| CaCO₃ | Alg-acid | KHCO₃ | Mannitol | Starch | Sugar | Talc | LMO | | 5.767 | 100 |
| CaCO₃ | Alg-acid | KHCO₃ | Mannitol | Starch | Sugar | Talc | LMO | NaHMP | 4.893 | 131 |

TABLE 9

| Simple Blend (Processed) | | | | | | | | | Raft | R&R test |
|---|---|---|---|---|---|---|---|---|---|---|
| | Alginic | | | | | | | | Strength | Raft in min. |
| CaCO3 | acid | KHC03 | Mannitol | Starch | Sugar | Talc | LMO | NaHMP | Force in | to reach |
| 500 mg | 200 mg | 140 mg | 800 mg | 71.43 mg | 654.8 mg | 27.68 mg | 15.08 mg | 4.55 mg | gram | to pH 4.0 |
| CaCO3* | Alg-acid | KHCO3 | | | | | | | 6.510 | 55 |
| CaCO3* | Alg-acid | KHCO3 | Mannitol | | | | | | 6.549 | 56 |
| CaCO3** | Alg-acid | KHCO3 | Mannitol | Starch | | | | | 5.402 | 198 |
| CaCO3** | Alg-acid | KHCO3 | Mannitol | | Sugar | | | | 7.354 | 200 |
| CaCO3** | Alg-acid | KHCO3 | Mannitol | | | Talc | | | 5.114 | 163 |
| CaCO3** | Alg-acid | KHCO3 | Mannitol | | | | LMO | | 4.047 | 163 |
| CaCO3** | Alg-acid | KHCO3 | Mannitol | | | | | NaHMP | 5.367 | 179 |
| CaCO3** | Alg-acid | KHCO3 | Mannitol | Starch | Sugar | | | | 5.788 | 179 |
| CaCO3** | Alg-acid | KHCO3 | Mannitol | Starch | Sugar | Talc | | | 7.430 | 205 |
| CaCO3** | Alg-acid | KHCO3 | Mannitol | Starch | Sugar | Talc | LMO | | 9.015 | 162 |
| CaCO3** | Alg-acid | KHCO3 | Mannitol | Starch | Sugar | Talc | LMO | NaHMP | 11.016 | 225 |

CaCO3*: granulated with water.
CaCO3**: granulated with starch, sugar, talc, LMO(liquid mineral oil) and NaHMP(sodium hexametaphosphate).

TABLE 10

| MB with various sugars | | | | | | | | Raft | R&R test |
|---|---|---|---|---|---|---|---|---|---|
| | Alginic | | | | | | | Strength | Raft in min. |
| MB | acid | KHCO₃ | Mannitol | Sorbitol | Xylitol | Dextrose | Fructose | Force in | to reach |
| 1293.6 mg | 200 mg | 140 mg | 800 mg | 800 mg | 800 mg | 800 mg | 800 mg | gram | to pH 4.0 |
| MB | Alg-acid | KHCO3 | Mannitol | | | | | 16.042 | 230 |
| MB | Alg-acid | KHCO3 | | Sorbitol | | | | 13.912 | 228 |
| MB | Alg-acid | KHCO3 | | | Xylitol | | | 16.777 | 244 |
| MB | Alg-acid | KHC03 | | | | Dextrose | | 15.966 | 218 |
| MB | Alg-acid | KHCO3 | | | | | Fructose | 19.728 | 249 |

### Experiment 6

### Addition of Polymers

Results from the R&R test provided data for duration in minutes and the Texture Analyzer provided data for force of the raft in grams. The following polymers were used in 200 mg/tablet dosage, replacing 200 mg mannitol: polyethylene oxide (as PolyOx), calcium polycarbophiphil, HPMC (medium viscosity), PEG 3350, sodium carboxymethylcellulose (Na CMC), and polyvinyl alcohol.

Addition of Polymers (600 mg mannitol and 200 mg polymer)

| | | | | | | Polyvinyl |
|---|---|---|---|---|---|---|
| Sample | Polyox | CaPoyCarbophil | HPMC(MV) | PEG335 [symbol] | NaCMC | alcohol |
| Duration in min.* | >164 | >164 | >198 | >198 | >196 | >200 |
| Strength (force in g) | 1 [symbol] | 1 [symbol] | 5. [symbol] | 2 [symbol] | | 1 [symbol] |

*Time for raft pH to reach 4.0, maximum time 200 minutes, value of shortest of two runs

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the are can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

1. A pharmaceutical composition for a chewable tablet, comprising in admixture:
an alginic acid or a salt thereof;
a water-soluble carbonate radical precursor;
a calcium salt;
a first bulk sweetener;
a binding agent; and
wherein said calcium salt and either or both of said first bulk sweetener and said binding agent are blended via wet granulation prior to admixture with said alginic acid or salt thereof and said carbonate radical precursor.

2. The chewable tablet according to claim 1, wherein said alginic acid or salt thereof is present in an amount from about 70 to about 500 mg per tablet.

3. The chewable tablet according to claim 2, wherein said alginic acid or salt thereof is present in an amount from about 200 to about 400 mg per tablet.

4. The chewable tablet according to claim 3, wherein said alginic acid or salt thereof is present in an amount from about 200 to about 300 mg per tablet.

5. The chewable tablet according to claim 1, wherein said carbonate radical precursor is selected from the group consisting of a carbonate of an alkali metal, a bicarbonate of an alkali metal, a carbonate of an alkaline earth metal, a bicarbonate of an alkaline earth metal, and combinations thereof.

6. The chewable tablet according to claim 5, further comprising an alkali metal or alkaline earth metal of hexametaphosphate, wherein said alkali metal is selected from the group consisting of sodium or potassium and said alkaline earth metal is selected from the group consisting of calcium, magnesium, and manganese.

7. The chewable tablet according to claim 5, wherein said carbonate radical precursor is selected from the group consisting of potassium bicarbonate, sodium bicarbonate, and a combination thereof.

8. The chewable tablet according to claim 7, wherein said carbonate radical precursor is present in an amount from about 50 to about 200 mg per tablet.

9. The chewable tablet according to claim 8, wherein said carbonate radical precursor is present in an amount from about 70 to about 160 mg per tablet.

10. The chewable tablet according to claim 1, wherein said calcium salt is selected from the group consisting of calcium citrate, calcium maleate, calcium citrate maleate, calcium carbonate, calcium lactate, calcium glyceryl phosphate, calcium phosphate, and combinations thereof.

11. The chewable tablet according to claim 10, wherein said calcium salt is calcium carbonate.

12. The chewable tablet according to claim 1, wherein said calcium salt is present in an amount from about 100 to about 1000 mg free calcium per tablet.

13. The chewable tablet according to claim 12, wherein said calcium salt is present in an amount from about 250 to about 1000 mg free calcium per tablet.

14. The chewable tablet according to claim 13, wherein said calcium salt is present in an amount of about 500 mg free calcium per tablet.

15. The chewable tablet according to claim 1, wherein said binding agent is selected from the group consisting of a starch, a natural gum, a low viscosity cellulosic derivative, a medium viscosity cellulosic derivative, a polymer, and combinations thereof.

16. The chewable tablet according to claim 15, wherein said binding agent is a starch selected from the group consisting of corn starch, modified corn starch, wheat starch, modified wheat starch, Starch 1500, pre-gelatinized starch, and combinations thereof.

17. The chewable tablet according to claim 16, wherein said starch is corn starch or modified corn starch.

18. The chewable tablet according to claim 15, wherein said starch is present in an amount from about 1% to about 15% of the tablet weight.

19. The chewable tablet according to claim 15, wherein the binding agent is a low-viscosity cellulosic derivative selected from the group consisting of carbomer, hydroxypropylmethylcellulose, methylcellulose, hydroxypropylcellulose, microcrystalline cellulose, carboxymethylcellulose, hydroxyethylcellulose, methylcellulose, and combinations thereof.

20. The chewable tablet according to claim 19, wherein said cellulosic derivative is present in an amount from about 1% to about 10% of the tablet weight.

21. The chewable tablet according to claim 15, wherein said binding agent is a natural gum selected from the group consisting of pectin, gelatin, gum arabic, acacia, carrageenan, guar, tragacanth, and combinations thereof.

22. The chewable tablet according to claim 21, wherein said natural gum is present in an amount from about 0.5% to about 7% of the tablet weight.

23. The chewable tablet according to claim 1, wherein said binding agent is selected from the group consisting of povidone, maltodextrin, mannitol, sorbitol, a polaxomer, a polydextrose, polyethylene glycol, a polymethacrylate, and combinations thereof.

24. The chewable tablet according to claim 1, wherein said binding agent is selected from the group consisting of polyethylene oxide, sodium carboxymethylcellulose, polyvinyl alcohol, calcium polycarbophil, HPMC (medium viscosity), and polyethylene glycol (PEG); or combinations thereof and/or combinations with other binding agents.

25. The chewable tablet according to claim 1, wherein said first bulk sweetener is a sugar selected from the group consisting of dextrose, sucrose, lactose, confectionery sugar, powdered sugar, dextrin, fructose, glucose, polydextrose, sorbitol, maltitol, maltose, mannitol, xylitol, and combinations thereof.

26. The chewable tablet according to claim 24, wherein said first bulk sweetener is a sugar selected from the group consisting of dextrose, sucrose, and a combination thereof.

27. The chewable tablet according to claim 1, wherein said first bulk sweetener is a polyol selected from the group consisting of mannitol, sorbitol, xylitol, maltitol, maltose, polydextrose, and combinations thereof.

28. The chewable tablet according to claim 27, wherein said polyol is selected from the group consisting of mannitol, sorbitol, and a combination thereof.

29. The chewable tablet according to claim 1, wherein said first bulk sweetener is wet granulated with said calcium salt, and wherein said first bulk sweetener is present in an amount from about 10% to about 30% of the tablet weight.

30. The chewable tablet according to claim 26, wherein said first bulk sweetener is a sugar wet granulated with said calcium salt, and wherein said sugar is present in an amount from about 15% to about 25% of the tablet weight.

31. The chewable tablet according to claim 1, wherein said first bulk sweetener is a sugar selected from the group consisting of dextrose; sucrose; lactose; confectionery sugar; powdered sugar; a polyol selected from the group consisting of mannitol, sorbitol, xylitol, erythritol, maltitol, maltose, polydextrose, and combinations thereof; and combinations thereof.

32. The chewable tablet according to claim 1, further comprising a second bulk sweetener, wherein said second bulk sweetener is a sugar selected from the group consisting of dextrose; sucrose; lactose; confectionery sugar; powdered sugar; a polyol selected from the group consisting of mannitol, sorbitol, xylitol, erythritol, maltitol, maltose, polydextrose, and combinations thereof; and combinations thereof.

33. The chewable tablet according to claim 32, wherein said second bulk sweetener is selected from the group consisting of dextrose, sucrose, lactose, confectionery sugar, powdered sugar, and combinations thereof.

34. The chewable tablet according to claim 32, wherein said second bulk sweetener is a polyol selected from the group consisting of mannitol, sorbitol, xylitol, erythritol, maltitol, maltose, polydextrose, and combinations thereof.

35. The chewable tablet according to claim 32, wherein said second bulk sweetener is a polyol selected from the group consisting of mannitol, sorbitol, and a combination thereof.

36. The chewable tablet according to claim 32, wherein said second bulk sweetener is a combination of dextrose and a polyol selected from the group consisting of mannitol, sorbitol, and a combination thereof.

37. The chewable tablet according to claim 32, wherein said second bulk sweetener is present in an amount from about 200 mg to about 1000 mg per tablet or 0.8% to 40% by weight of the tablets.

38. The chewable tablet according to claim 1, further comprising a second bulk sweetener, and wherein said first bulk sweetener and said second bulk sweetener are not the same; and further wherein a portion of the first or second bulk sweetener is optionally replaced by gelatin or casein.

39. The chewable tablet according to claim 1, wherein said first bulk sweetener is selected from the group consisting of sucrose, mannitol, dextrose, and combinations thereof and the second bulk sweetener is selected from the group consisting of mannitol, sorbitol, dextrose, and combinations thereof.

40. The chewable tablet according to claim 1, wherein both said binding agent and said first bulk sweetener are blended with the calcium salt by wet granulation.

41. The chewable tablet according to claim 40, wherein said binding agent is present in an amount from about 1% to about 15%, wherein said first bulk sweetener is present in an amount from about 10% to about 30%, and wherein said calcium salt is present in an amount from about 10% to about 50% by weight of tablet.

42. The chewable tablet according to claim 41, wherein said binding agent is corn starch, wherein said first bulk sweetener is sucrose, and wherein said calcium salt is calcium carbonate.

43. The chewable tablet according to claim 1, further comprising talc, wherein said talc is present in an amount from about 0.5% to about 3% of the tablet weight.

44. The chewable tablet according to claim 1, further comprising an intense sweetener, wherein said intense sweetener is selected from the group consisting of acesulfame-K, saccharin, aspartame, sucralose, and combinations thereof.

45. The chewable tablet according to claim 1, further comprising an intense sweetener, wherein said intense sweetener is present in an amount from about 0.02% to about 0.12% of the tablet weight.

46. The chewable tablet according to claim 1, further comprising a mineral oil, wherein the mineral oil is present in an amount up to about 1% of the tablet weight.

47. The chewable tablet according to claim 1 wherein a portion of the water soluble carbonate radical precursor is replaced with sodium or potassium phosphate.

48. A method of calcium supplementation to a mammal in need thereof, comprising administering an effective amount of a composition according to any one of claims 1 to 47.

49. A method of reducing gastric reflux in a human in need thereof, comprising administering to said human an effective amount of a composition according to claim 1.

50. A method of reducing heartburn in a human in need thereof, comprising administering to said human an effective amount of a composition according to claim 1.

51. A method of reducing the incidence of gastric reflux in the esophageal cavity in a human for a period of time following post ingestion of a meal causing gastric reflux in said human for a time period of about 60 to about 480 minutes, comprising administering to said human an effective amount of a composition according to claim 1.

52. The method according to claim 51, wherein said time period is from about 120 to about 300 minutes.

53. The method according to claim 52, wherein the time period is about 120 to 180 minutes

54. The method according to claim 51 wherein the pH of the esophageal cavity is maintained at a pH of about 4.0 or higher.

55. A method of maintaining a pH of about 4.0 or higher in the esophageal cavity of a human for a time period from about 60 to about 480 minutes, comprising: administering to said human an effective amount of a composition according to claim 1.

56. The method according to claim 55, wherein said pH is 5.0 or higher.

57. The method according to claim 55, wherein said time period is from about 120 to about 300 minutes.

58. The method according to claim 57 wherein said time period is about 120 to 180 minutes.

59. A pharmaceutical composition comprising calcium carbonate, sugar, mannitol, corn starch, alginic acid, and a carbonate radical precursor selected from the group consisting of potassium bicarbonate, sodium bicarbonate and a combination thereof, wherein the composition is in the form of a chewable tablet.

60. The composition according to claim 59, wherein a portion of the carbonate radical precursor is replaced by sodium or potassium phosphate.

61. The composition according to claim 59, wherein said alginic acid is present in an amount of about 8%; wherein said potassium bicarbonate is present in an amount of about 6%; wherein said calcium carbonate in present in an amount of about 20% w/w; wherein said starch is present in amount about 5%; wherein said sugar is present in an amount about 26%; and wherein said mannitol is present in an amount of about 32% by weight of the tablet.

62. The composition according to claim 61, further comprising an intense sweetener, a talc, a light mineral oil, sodium hexametaphosphate, and calcium stearate, wherein said intense sweetener is present in an amount of about 0.1%, wherein said talc is present in an amount about 1%, wherein said light mineral oil present in an amount about 0.6%, wherein said sodium hexametaphosphate is present in an amount about 0.2%, and wherein said calcium stearate present in an amount of about 0.5% by weight of the tablet.

63. The composition according to claim 1, wherein said carbonate radical precursor is a compound different than that of said calcium salt.

64. A pharmaceutical composition for a chewable tablet formed by a process comprising the following steps:
providing an alginic acid or a salt thereof;
providing a water-soluble carbonate radical precursor;
providing a calcium salt;
providing a first bulk sweetener;
providing a binding agent;
mixing said calcium salt and either or both of said bulk sweetener and said binding agent via wet granulation to form a mixture; and
blending said mixture with said alginic acid or salt thereof, said carbonate radical precursor, and with either said first bulk sweetener or said binding agent if not previously mixed with the calcium salt.

65. The composition of claim 64, wherein said alginic acid or salt thereof is provided in an amount from about 70 to about 500 mg per tablet.

66. The composition of claim 64, wherein said carbonate radical precursor provided is selected from the group consisting of a carbonate of an alkali metal, a bicarbonate of an alkali metal, a carbonate of an alkaline earth metal, a bicarbonate of an alkaline earth metal, and combinations thereof.

67. The composition of claim 66, wherein said carbonate radical precursor provided is selected from the group consisting of potassium bicarbonate, sodium bicarbonate, and a combination thereof.

68. The composition of claim 66, wherein said carbonate radical precursor is partially replaced by sodium or potassium phosphate in a % w/w amount.

69. The composition of claim 67, wherein said carbonate radical precursor is provided in an amount from about 50 to about 200 mg per tablet.

70. The composition of claim 64, wherein said calcium salt provided is selected from the group consisting of calcium citrate, calcium maleate, calcium citrate maleate, calcium carbonate, calcium lactate, calcium glyceryl phosphate, calcium phosphate, and combinations thereof.

71. The composition of claim 64, wherein said calcium salt provided is calcium carbonate.

72. The composition of claim 64, wherein said calcium salt is provided in an amount from about 100 to about 1000 mg free calcium per tablet.

73. The composition of claim 64, wherein said binding agent provided is selected from the group consisting of a starch, a polymer, a natural gum, a low viscosity cellulosic derivative, a medium viscosity cellulosic derivative, and combinations thereof.

74. The composition of claim 64, wherein said first bulk sweetener provided is a sugar selected from the group consisting of dextrose, sucrose, lactose, confectionery sugar, powdered sugar, dextrin, fructose, glucose, polydextrose, sorbitol, malititol, maltose, mannitol, xylitol, and combinations thereof; and further wherein the a portion of the first bulk sweetener is optionally replaced by gelatin or casein.

75. The composition of claim 64, wherein said first bulk sweetener provided is in an amount about 10% to about 30% of the tablet weight.

76. The composition of claim 64, which further comprises the addition of magnesium or aluminum cation in the form of an antacid.

77. The composition of claim 76, wherein the magnesium or aluminum antacid is selected from the group consisting of magnesium carbonate, magnesium oxide, magnesium hydoxide, magnesium aluminate, aluminum hydroxide, or aluminum magnesium hydroxide; or combinations thereof.

78. A pharmaceutical composition for a chewable tablet, comprising in admixture:
an alginic acid or a salt thereof;
a water-soluble carbonate radical precursor;
a calcium salt;
a first bulk sweetener; and
a binding agent.

79-216. (canceled)
